(19) United States
(12) Patent Application Publication
(10) Pub. No.: US 2024/0019440 A1
(43) Pub. Date: Jan. 18, 2024

(54) MULTIPLE ANALYTE FECAL ANTIGEN TESTING

(71) Applicant: Exact Sciences Corporation, Madison, WI (US)

(72) Inventors: Keith D. Fourrier, Madison, WI (US); Michael J. Domanico, Middleton, WI (US); Stephanie M. Weber, Madison, WI (US); Tanya M. Quint, Waunakee, WI (US); Graham P. Lidgard, Middleton, WI (US)

(21) Appl. No.: 17/769,533

(22) PCT Filed: Oct. 16, 2020
(86) PCT No.: PCT/US2020/056086

§ 371 Date: Apr. 15, 2022

Related U.S. Application Data
(60) Provisional application No. 62/923,300, filed on Oct. 18, 2019.

(51) Int. Cl. G01N 33/574

(52) U.S. Cl. CPC G01N 33/57419

(57) ABSTRACT

Methods, compositions, and systems for testing fecal samples, including small human fecal samples, for a plurality of different marker analytes, e.g., a plurality of different human protein, nucleic acid, and other molecular marker analytes.

The present application claims priority to U.S. Provisional Application Ser. No. 62/923,300, filed Oct. 18, 2019, which is incorporated herein by reference.

### FIELD OF THE INVENTION

Provided herein are methods, compositions, and systems, e.g., sets of articles, for testing fecal samples, including small human fecal samples, e.g., samples collected in Fecal Immunochemical Test (FIT) sample collection devices, for a plurality of different protein, nucleic acid, and other molecular marker analytes.

### BACKGROUND OF THE INVENTION

Fecal immunochemical tests (FIT tests) are antibody-based assays that detect hemoglobin in a stool specimen and are widely used for colorectal cancer (CRC) screening. Several different laboratory-based or point-of-care FIT tests are available. Available FIT tests typically use special devices to collect a small sample from a stool specimen, and to introduce the sample into a buffer contained within the device to stabilize hemoglobin from the time of sample collection until the sample can be assayed. FIT is a convenient and affordable option for CRC screening.

While other protein biomarkers that may be associated with colorectal adenoma and cancer have been identified in stool by screening relatively large specimens using electrophoresis and mass spectrometry (see, e.g., U.S. Patent Publ. 2015/0141273 A1 and 2017/0199196 A1 to Bosch, both incorporated herein by reference for all purposes), laboratory-based and point-of-care FIT tests remain limited to hemoglobin testing.

### SUMMARY OF THE INVENTION

The technology provides methods and compositions for collecting and stabilizing and protein biomarkers other than, or in addition to, hemoglobin, using FIT sample collection device. Accordingly, in some embodiments the technology provides a method of characterizing a human fecal sample, comprising i) providing a dispersed sample comprising a small human fecal sample dispersed in a volume of a stabilizing solution, and ii) assaying the dispersed sample for amounts of a plurality of different marker analytes. As used herein, different marker analytes may comprise different portions of loci of a single molecule, e.g., different fragments or regions of a protein or nucleic acid analyte molecule. In certain embodiments, different marker analytes are different completely different molecules, e.g., distinct proteins and/or distinct genes or other nucleic acids. In preferred embodiments, plurality of different marker analytes comprises a plurality of different protein marker analytes, preferably comprising at least three different protein marker analytes.

The plurality of different marker analytes may be, for example, proteins expressed from three different genes. In certain embodiments, the plurality of different marker analytes comprises no more than three different marker analytes. In some embodiments, the plurality of different marker analytes comprises human marker analytes, e.g., human protein and/or nucleic acid analytes. In preferred embodiments, the plurality of different marker analytes consists of human marker analytes.

The technology herein finds use in analysis of small fecal samples, e.g., fecal samples collected using a FIT sample collection device, e.g., a FIT sample collection device that comprises a fluid for stabilizing a small fecal sample. Accordingly, in some embodiments, the volume of stabilizing solution in the dispersed sample is preferably a volume suitable for use in a FIT sample collection device, e.g., less than 3 mL, preferably less than 2 mL. In some embodiments, the stabilizing solution has a volume of less than 1 mL. In some embodiments, the dispersed sample is provided in a FIT sample collection device.

Similarly, while the mass of fecal sample for analysis is not limited to any particular mass, in certain embodiments, the mass of a fecal sample is that of a sample collectable using a FIT sample collection device. Accordingly, in certain embodiments, the small fecal sample has a mass of less than 100 mg, preferably less than 50 mg, preferably less than 20 mg.

The technology is not limited to any particular marker analytes, e.g., for detection in the dispersed sample. For example, in some embodiments, the plurality of different marker analytes comprises a plurality of human protein marker analytes selected from the group consisting of hemoglobin (Hb), calprotectin, haptoglobin (Hp), complement component 3 (C3), complement component 3a (C3a), Fc fragment of IgG binding protein (FCGBP), resistin like beta (RETNLB/RELM), S100 calcium binding protein A12 (S100A12), and serpin family F member 2 (SERPIN F2). In some embodiments, the plurality of different human protein marker analytes comprises Hb, Hp, and C3, and in some embodiments, the plurality of different human protein marker analytes consists of Hb, Hp, and C3.

While the technology is not limited to any particular method of assaying the marker analytes, in certain embodiments, the assaying comprises an immunochemical assay.

In some embodiments, a stabilizing solution comprises a buffer, e.g., a tris(hydroxymethyl)aminomethane (Tris) buffer. Other components may also be used in the stabilizing solution, e.g., to stabilize cells, stabilize proteins, retard microbial growth, etc., Accordingly, the technology contemplates stabilizing solutions comprising one or more components selected from a buffer, a carrier protein, a detergent, a salt, a chelator, and an antimicrobial or antibiotic. In certain embodiments, a stabilizing solution comprises one or more of tris(hydroxymethyl)aminomethane (Tris) buffer; bovine serum albumin; polyethylene glycol sorbitan monolaurate (TWEEN-20); sodium azide; sodium chloride; ethylenediaminetetraacetic acid (EDTA); and gentamicin. In certain preferred embodiments, a stabilizing solution comprises 20 mM Tris, 10% bovine serum albumen, 0.10% TWEEN 20, 0.095% sodium azide, 140 mM sodium chloride, 10 mM EDTA, and 15 μg/ml gentamicin.

Other stabilizing components may also be used, e.g., in a stabilizing solution as discussed above, or in a solution having a different composition. In certain embodiments, components suitable for stabilizing hemoglobin may be included. For example, in some embodiments, the stabilizing solution comprises one or more stabilization reagents selected from: a protoporphyrin; a polyvalent cation; a sugar or polysaccharide; an osmolyte and optionally, a polyvalent cation; a horse radish peroxidase (HRP) stabilization component and, optionally, a polyvalent cation. IN certain embodiments, the stabilizing solution comprises protoporphyrin IX complexed with a multivalent cation, preferably a multivalent cation selected from Cr³⁺ and Co³⁺. In some embodiments, the stabilizing solution comprises a substituted or unsubstituted polygalacturonic acid and, optionally, a polyvalent cation.

It is contemplated that an amount of a stool sample remaining after removal of a small fecal sample may also be assayed for one or more marker analytes. Accordingly, in some embodiments, the technology provides any of the embodiments described herein, wherein the small human fecal sample is provided by a method comprising a) collecting a whole stool sample from a human subject; b) removing a portion of the whole stool sample to produce a removed portion and a remaining portion of the stool sample, wherein the removed portion is a small fecal sample; c) combining the small fecal sample with the volume of stabilizing solution to produce the dispersed sample; and d) stabilizing the remaining portion of the stool sample.

The remaining portion of the stool sample may be stabilized in a number of different ways, For example, in some embodiments, stabilizing the remaining portion of the stool sample comprises one or both of i) adding a stabilizing buffer to the remaining portion of the stool sample, preferably homogenizing the remaining portion of the stool sample in a stabilizing buffer to form a stool homogenate; ii) freezing the remaining portion of the stool sample. In certain preferred embodiments, the stabilizing buffer comprises about 100 to about 300 mM of a chelating agent, preferably a chelating agent comprising EDTA, and comprises between about 400 and about 600 mM of Tris hydrochloride.

In some embodiments, the assaying the remaining portion of the stool sample comprises processing a stool homogenate to separate solids from a clarified fluid fraction and assaying the clarified fluid fraction. Means of producing a clarified fluid fraction may include, e.g., filtration, e.g., to produce a clarified filtrate, and/or centrifugation, e.g., to produce a clarified supernatant. Methods of processing stool samples, removing assay inhibitors, and isolating target nucleic acid molecules include, for example, methods disclosed in U.S. Pat. No. 10,047,390, which is incorporated herein by reference for all purposes.

For analysis of the remaining portion of the stool sample, one or more marker analytes assayed in the remaining portion of the stool sample may comprise a marker analyte or a plurality of analytes that are also assayed in the dispersed sample. For example, in some embodiments, the dispersed sample and the remaining portion of the stool sample are assayed for the same set of marker analytes. In other embodiments, the one or more marker analytes assayed in the remaining portion of the stool sample consists of marker analytes not assayed in the dispersed sample.

In some embodiments, the marker analytes assayed in the remaining portion of the stool sample comprises one or more human DNA marker analytes, preferably one or more human DNA marker analytes that are assayed for at least one of a mutation and a methylation status of a cytosine. Examples of human DNA marker analytes assayable using the methods, compositions, and sets of articles described herein include, for example, markers described in WO 2015/153289 (PCT/US2015/022749), which is incorporated herein by reference in its entirety. It is contemplated that the dispersed sample may similarly be analyzed for one or more the DNA marker analytes, or for other nucleic acid analytes.

The technology also provides articles, e.g., containers, collection devices, reagents, and the like, for characterizing a human fecal sample using any one of the methods set forth above. For example, in some embodiments, the technology provides set of articles comprising a FIT sample collection device containing a dispersed sample comprising a small human fecal sample dispersed in a volume of a stabilizing solution, and a set of reagents for assaying the dispersed sample for amounts of a plurality of different human protein marker analytes. In preferred embodiments, the volume of stabilizing solution in the FIT sample collection device is less than 3 mL, preferably less than 2 mL.

In some embodiments, the plurality of different human protein marker analytes assayable by the set of reagents comprises at least three different protein marker analytes, and in certain preferred embodiments, the plurality of different human protein marker analytes comprises no more than three different marker analytes. Preferably, the plurality of different human protein marker analytes is selected from the group consisting of Hb, calprotectin, Hp, C3, C3a, FCGBP, RETNLB/RELM) S100A12, and SERPIN F2, preferably comprising Hb, Hp, and C3. In certain embodiments, the plurality of different human protein marker analytes assayable by the set of reagents consists of Hb, Hp, and C3. While the technology is not limited to particular assay methods, in certain embodiments, the set of reagents for assaying the dispersed sample comprises a set of reagents for immunochemical assays.

While the mass of the human fecal sample is not limited to a particular size, in some embodiments the small human fecal sample has a mass of less than 100 mg, preferably less than 50 mg, preferably less than 20 mg. Any suitable stabilizing solution, e.g., any of the stabilizing solutions and variations thereof discussed above, may be used in the set of articles.

In some embodiments of the set of articles wherein the small human fecal sample comprises a portion removed from a whole stool sample, the set of articles further comprises a stabilized remaining portion of the stool sample. Preferably, the stabilized remaining portion of the stool sample comprises a stabilizing buffer. Any suitable stabilizing buffer, e.g., any of the stabilizing buffers and variations thereof discussed above, may be used in the set of articles. In some embodiments, the stabilized remaining portion of the stool sample is homogenized in the stabilizing buffer.

The set of articles may further comprise a set of reagents for assaying the stabilized remaining portion of the stool sample for amounts of one or more marker analytes. In some embodiments, the one or more marker analytes assayable by the set of reagents for assaying the stabilized remaining portion of the stool sample comprises at least one human protein marker analyte assayable by the set of reagents for assaying the dispersed sample, and in some embodiments, the one or more marker analytes assayable by the set of reagents for assaying the stabilized remaining portion of the stool sample comprises one or more human DNA marker analytes. In preferred embodiments, set of reagents for assaying the stabilized remaining portion of the stool sample for one or more human DNA marker analytes comprises reagents for assaying at least one of a mutation; and a methylation status of a cytosine.

### DEFINITIONS

To facilitate an understanding of the present technology, a number of terms and phrases are defined below. Additional definitions are set forth throughout the detailed description.

As used herein, “a” or “an” or “the” can mean one or more than one. For example, “a” widget can mean one widget or a plurality of widgets.

The terms “fecal” and “stool” are used interchangeably herein in reference to samples of feces, e.g., human feces.

As used herein, the term “metered” means having a reasonably reproducible measured quantity.

As used herein, the term “FIT sample collection device” refers to a device for collecting a fecal sample, preferably for collecting a metered fecal sample, for analysis. See, e.g., U.S. Pat. Nos. 9,211,112; 7,780,915; and 6,780,160. In some embodiments, a FIT sample collection device comprises an amount of a stabilizer, e.g., a stabilizing buffer, in an amount suitable for stabilizing components in an amount of a fecal sample collected by the FIT sample collection device.

As used herein, the term “FIT device sample” refers to small fecal sample collected by a FIT sample collection device. A FIT device sample is preferably a small fecal sample as defined herein below, and is more preferably a metered sample. In some embodiments, a FIT device sample comprises the small fecal sample in combination with an amount of a fluid, e.g., a stabilizing buffer, in an amount suitable for stabilizing one or more components of the sample, e.g., protein, nucleic acid, carbohydrate, or other components of a fecal sample.

As used herein, “small fecal sample” refers to a fecal sample of less than about 100 mg, preferably less than 90 mg, preferably less than 80 mg, preferably less than 70 mg, preferably less than 60 mg, preferably less than 50 mg, preferably less than 40 mg, preferably less than 30 mg, preferably less than 20 mg, preferably less than 10 mg, preferably less than 9 mg, preferably less than 8 mg, preferably less than 7 mg, preferably less than 6 mg, preferably less than 5 mg, preferably less than 4 mg, preferably less than 2 mg, preferably less than 1 mg, including any integer value of a microgram and any fraction of a microgram therebetween. In preferred embodiments, a small fecal sample is combined with a volume of a stabilizing fluid, e.g., a stabilizing buffer suitable for stabilizing one or more components, preferably macromolecular components, e.g., protein, nucleic acid, carbohydrate, and other components of a fecal sample.

As used herein, the term “whole stool sample” refers to an undivided product of a bowel movement by a subject, e.g., as collectable by defecation by the subject directly onto or into a stool collection device, e.g., a vessel, container, or surface.

As used herein, the term “dispersed sample” refers to a sample (e.g., a fecal sample) in combination with a dispersion matrix, (e.g., fluid, gel, etc.), wherein sample material is distributed within the matrix. A dispersed sample need not be mixed to uniformity in a matrix. For example, sample material may be mixed, e.g., by homogenization, such that all sample present is distributed essentially uniformly throughout the matrix, or mixing may result in a fraction of an original sample material remaining in a distinguishable form, e.g., a residual semi-solid portion of a fecal sample, in the presence of matrix comprising a dispersed fraction of the sample.

The terms “stabilizing solution” and “stabilizing buffer” are used interchangeably herein in reference to a fluid matrix suitable for stabilizing one or more components, preferably macromolecular components, e.g., protein, nucleic acid, carbohydrate, and other components, of a fecal sample. Stabilizing buffers include but are not limited to buffers selected to stabilize proteins (e.g., hemoglobin) or nucleic acids (e.g., DNA or RNA) or to stabilize both in a sample, e.g., a blood or stool sample. In some embodiments, a stabilizing buffer comprises a protein stabilizing buffer as described, e.g., in U.S. Patent Publication 2019/0302129 A1; PCT Application Ser. No. PCT/US2019/022598; and U.S. Pat. No. 9,211,112; or a nucleic acid stabilizing buffer as described, e.g., in U.S. Pat Nos. 6,551,777; 8,722,330, 10,064,404; 9,376,709; 9,657,227; and 9,926,590; 10,144,955, each of which is incorporated herein by reference in its entirety.

Stool stabilizing buffers for FIT sample collection devices comprising one or more hemoglobin stabilization reagents are contemplated. In some embodiments, the one or more hemoglobin stabilization reagents may be selected from, e.g., an osmolyte; a polyvalent cation; a sugar or polysaccharide and, optionally, a polyvalent cation; a protoporphyrin; and a horse radish peroxidase (HRP) stabilization component and, optionally, a polyvalent cation. Examples and concentrations of such reagents in the solution are set forth US 2019/0302129 A1.

In some embodiments, the solution may comprise an osmolyte, e.g., betaine. In these 15 embodiments, the osmolyte (e.g., betaine) may be at a concentration in the range of 2 M to 5 M.

In some embodiments, the solution may comprise a sugar, e.g., sucrose or trehalose. In these embodiments, the sugar (e.g., sucrose or trehalose) may be at a concentration in the range of 0.1 M to 0.5 M. In either case, the solution may optionally contain a polyvalent cation, Mg²⁺ or Ca²⁺. In these embodiments, the polyvalent cation (e.g., calcium or magnesium ions) may be at a concentration in the range of 5 mM to 25 mM.

In some embodiments, the solution may comprise a polysaccharide, e.g., a substituted or unsubstituted polygalacturonic acid such as α-(1-4)-linked D-galacturonic acid. In these embodiments, the polysaccharide (e.g., the substituted or unsubstituted polygalacturonic acid) may be at a concentration in the range of 0.005% to 0.5% (e.g., 0.01% to 0.125%). In these embodiments, the solution may optionally contain a polyvalent cation. In embodiments in which the solution contains a polyvalent cation, the polyvalent cation may be at a concentration in the range of 5 mM to 25 mM. In some embodiments, the solution may comprise substituted or unsubstituted polygalacturonic acid and a multivalent cation (e.g., a calcium salt or magnesium salt) at a concentration in the range of 5 mM to 25 mM.

In some embodiments, the solution may comprise a protoporphyrin, e.g., a protoporphyrin IX or an analog thereof such as octaethylporphyrin (H2OEP) or tetraphenylporphyrin (H2TPP), complexed with a metal ion. In these embodiments, the protoporphyrin may be hemin (protoporphyrin IX containing a ferric iron (Fe3+) ion with a coordinating chloride ligand) or hematin. In other embodiments, the protoporphyrin may be protoporphyrin IX complexed with a divalent or trivalent cation (e.g., Zn2+, Cr3+, or Co3+, for example). In these embodiments, the protoporhyrin may in the solution at a concentration in the range of 0.1 μM to 100 μM (e.g., 1 μM to 10 μM). In some embodiments, the solution may comprise an HRP stabilization component selected from HRP Conjugate Stabilizer (PN 85R-102; Fitzgerald Industries), HRP Conjugate Stabilizer (PN SZ02; Surmodics) and HRP Conjugate Stabilizer (PN ab171537; Abcam). Other HRP stabilization components, e.g., AbGuard (BioRad PN: BUF052; BioRad Laboratories Inc. 2000 Alfred Nobel Dr, Hercules, CA 94547) can potentially be used. If the solution comprises an HRP stabilization component, then the component may be at a concentration in the range of 1% to 20%, e.g., 5% to 15% or 5% to 20%.

In any embodiment, the solution may comprise a polyvalent cation, e.g., calcium or magnesium ions. In these embodiments, the polyvalent cation may be at a concentration in the range of 5 mM to 25 mM. In any embodiment, the solution further comprises tris(hydroxymethyl)aminomethane (Tris) buffer (e.g., 10 mM to 50 mM Tris, pH 7.5), bovine serum albumen (e.g., 5% to 20% BSA), polysorbate 20 (e.g., 0.05% to 0.2% polyoxyethylenesorbitan monolaurate (TWEEN® 20)), a preservative such as sodium azide (e.g., 0.05% to 0.2% sodium azide), a salt such as sodium chloride (e.g., 50 mM to 250 mM sodium chloride), a chelator such as ethylenediaminetetraacetic acid (EDTA; e.g., 5 mM to mM ethylenediaminetetraacetic acid), and an antibiotic such as gentamicin (e.g., 5 ug/mL to 50 ug/mL).

As used herein, the term “marker analyte” is used in its broadest sense, referring to any component or feature of a sample that can be analyzed or assayed to characterize the sample, e.g., for indication of a disease or condition, or absence of a disease or condition.

### DESCRIPTION OF THE DRAWINGS

FIG. 1 provides a table (Table 2) showing results of testing for the presence of a list of the protein markers in whole stool supernatant samples from subjects classified by tissue pathology as having colorectal cancer (CA), advanced adenoma (AA), or being negative for cancer and advanced adenoma.

FIG. 2 provides a table (Table 3) for the presence of a list of the protein markers in FIT device samples from subjects classified by tissue pathology as having colorectal cancer (CA), advanced adenoma (AA), or being negative for cancer and advanced adenoma.

### DETAILED DESCRIPTION OF THE INVENTION

Provided herein is technology for analysis of a plurality of markers in fecal samples, e.g., a plurality of different protein markers, with or without analysis of hemoglobin. In some embodiments a plurality of different markers is tested in a supernatant sample from a whole stool specimen, while in some embodiments, a plurality of different protein markers is tested in a small sample, e.g., as would be collected using a FIT sample collection device. FIT sample collection devices typically collect less than 0.1 g of sample, more typically between 10 and 20 mg of sample. In yet other embodiments, results from testing a plurality of proteins in a whole stool sample are combined with results of testing a sample from the same stool specimen collected using a FIT sample collection device. In yet other embodiments, the stool specimen is further tested for non-protein markers, e.g., DNA markers. The discussion below makes reference to the following abbreviations: hemoglobin (Hb) calprotectin, haptoglobin (Hp), complement component 3 (C3), complement component 3a (C3a), Fc fragment of IgG binding protein (FCGBP), resistin like beta (RETNLB/RELM), S100 calcium binding protein A12 (S100A12), and serpin family F member 2 (SERPIN F2).

All known Fecal Immunochemical Tests (FIT) only measure hemoglobin in the stool sample. Examples include:
1. CAREprime Hb (Alfresa Pharma, Osaka, Japan)
2. Hb ELISA (Immundiagnostik, Bensheim, Germany)
3. OC Sensor (Eiken Chemical, Tokyo, Japan) (Sold as Polymedco in the United States)
4. RIDASCREEN Hb (R-Biopharm, Darmstadt, Germany)
5. SENTiFIT-FOB Gold (Sentinel Diagnostics, Milan, Italy)
6. Eurolyser FOB test (Eurolyser Diagnostics, Salzburg, Austria)
7. ImmoCARE-C (CARE Diagnostics, Voerde, Germany)
8. QuantOn Hem (Immundiagnostik, Bensheim, Germany)
9. QuikRead go iFOBT (Orion Diagnostica, Espoo, Finland)

Anton Gies, et al., evaluated nine Fecal Immunochemical Tests for colorectal cancer. The tests were evaluated on the same sample set and compared. The results of the comparison indicate that when a specificity of ≥93% is set for all tests, almost equal sensitivities for CRC detection are observed. However, the test characteristics of each test demonstrate that at a specificity of 93%, none of the tests achieved a sensitivity >82% for CRC detection, and a specificity of 97%, none of the tests achieved a sensitivity of >75% for CRC detection in this data set. (Anton Gies et al. Direct Comparison of Diagnostic Performance of 9 Quantitative Fecal Immunochemical Tests for Colorectal Cancer Screening. Gastroenterology 2018; 154:93-104.) Conversely, while the sensitivity of these tests can be adjusted upward by varying the cutoffs, increased sensitivity comes at the expense of specificity.

One difficulty in testing different protein analytes is that protein stability in stool samples can be poor. Low analyte concentration and poor assay sensitivity can further limit meaningful detection and analysis of proteins in stool, especially in the small portions of stool typically collected by FIT sample collection devices. For example, the sample collection devices for the FIT tests listed above are configured to deliver a small, metered amount—typically 20 mg or less—for testing. Typically, these devices are configured to exclude excess sample, e.g., by scraping a sampling spoon or rod, such that a metered amount of the stool sample can be delivered to a reservoir or container having a set volume of a buffer, as shown in the table below:

| FIT Test Brand | Fecal mass collected | Buffer volume |
|---|---|---|
| CAREprime Hb | 10 mg | 1.9 mL |
| Hb ELISA | 15 mg | 1.5 mL |
| OC Sensor | 10 mg | 2 mL |
| RIDASCREEN Hb | 10 mg | 2.5 mL |
| SENTIFIT-FOB Gold | 10 mg | 1.7 mL |
| Eurolyser FOB test | 19.9 mg | 1.6 mL |
| ImmoCARE-C | 20 mg | 2.5 mL |
| QuantOn Hem | 15 mg | 1.5 mL |
| QuikRead go iFOBT | 10 mg | 2.0 mL |

(Gies, et al., supra)

While each of the FIT collection devices described above has been shown to deliver a sufficient amount of sample for detection of hemoglobin, detection of other protein markers in these same small samples has proven more challenging. Of the 24 protein markers evaluated only two markers demonstrated feasibility in improving the sensitivity and specificity of the FIT for Hb assay.

### EXPERIMENTAL EXAMPLES

FIT and whole stools samples were collected using the devices according to a COLOGUARD Collection Kit (Exact Sciences, Madison, WI). FIT samples were collected using a device and stabilized in a buffer as described in U.S. Pat. No. 9,211,112, with the resulting suspended sample tested directly.

Whole stool samples were collected and stabilized in Cologuard Stool Buffer 200204, comprising ethylenediaminetetraacetic acid (EDTA) and tris(hydroxymethyl)aminomethane (Tris) buffer. The stool samples were weighed, then combined with buffer. Preferably, buffer and stool are combined at a 4:1 (w:w or v:w) buffer-to-stool ratio. The samples are homogenized in the buffer, centrifuged to remove solids, and the supernatant is collected for biomarker testing.

Whole stool supernatant and FIT samples were evaluated for the presence/detection of 24 additional protein markers. For the evaluation, commercially available assay kits were purchased from several different vendors. A list of the protein marker assays that were used in the evaluation can be found in Table 1.

TABLE 1
List of Protein Biomarker Assays.

| Protein Biomarker | Vendor | Catalog # |
|---|---|---|
| Hemoglobin | Exact Sciences | NA |
| Complement C3a desArg | abcam | ab133037 |
| Human Complement C3 | abcam | ab108823 |
| Human Haptoglobin | abcam | ab108858 |
| Human LY6G6D | AVIVA Systems | OKCA00716 |
| | Biology | |
| Human Calprotectin | Eagle Biosciences, Inc | CAL35-K01 |
| Human Secretory IgA (sIgA) | Eagle Biosciences, Inc | SGA35-K01 |
| Human C5a | HycultBiotech | HK349-02 |
| Human C3a | Invitrogen | BMS2089 |
| Human CLDN1 | LifeSpan BioSciences | LS-F20989 |
| Human Complement C3a | LifeSpan BioSciences | LS-F25982 |
| Human FCGBP | LifeSpan BioSciences | LF-F815 |
| Human LGALS1 | LifeSpan BioSciences | LS-F26484 |
| Human LGALS3 | LifeSpan BioSciences | LS-F442 |
| Human Lysophosphatidic Acid | LifeSpan BioSciences | LS-F25111 |
| (LPA) | | |
| Human PIGR | LifeSpan BioSciences | LS-F23436 |
| Human PIGR | LifeSpan BioSciences | LS-F23436 |
| Human RETNLB/RELM-Beta | LifeSpan BioSciences | LS-F8002 |
| Human S100A12 | LifeSpan BioSciences | LS-F4417 |
| Human VWA2 | LifeSpan BioSciences | LS-F19358 |
| Human GPR56 | RayBiotech | ELH-GPR56 |
| Human LRRC4 | RayBiotech | ELH-LRRC4 |
| Human Serpin F2 | RayBiotech | ELH- |
| | | SERPINF2 |
| CA 19-9 | Roche (Cobas) | |
| CEA | Roche (Cobas) | |

Samples were tested according to manufacturer's instructions, with the volume of fluid tested depending on the different assay requirements. Across all kits, sample volume varied from 25-100 μL. For some procedures, the samples from the whole stool supernatants required dilution prior to testing, e.g., to reduce inhibition by the sample stabilization buffer and/or to produce a sample having target analytes within the measurement range of quantitative assays. Dilutions ranged from undiluted to 150-fold dilution. FIT collection device samples were typically diluted 10-fold prior to testing.

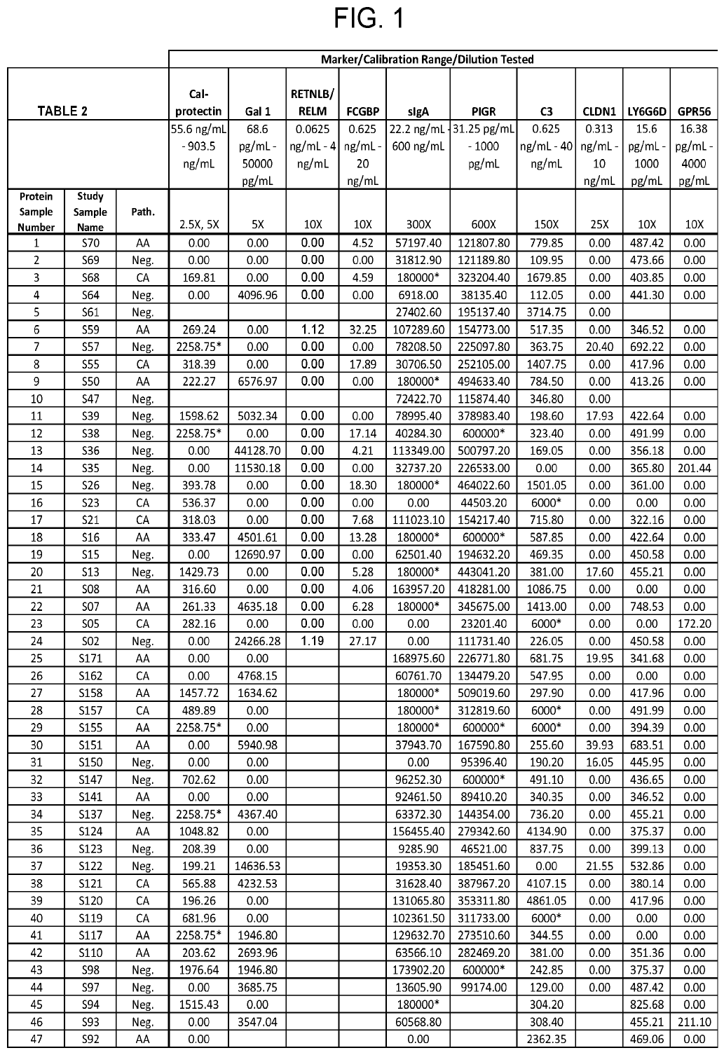

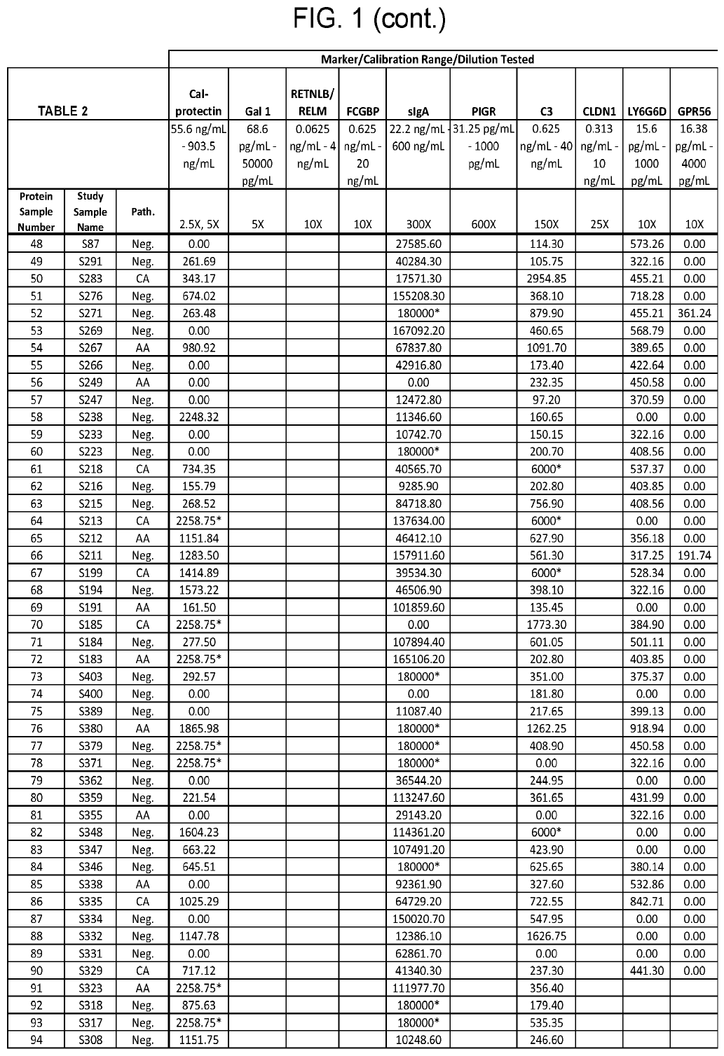

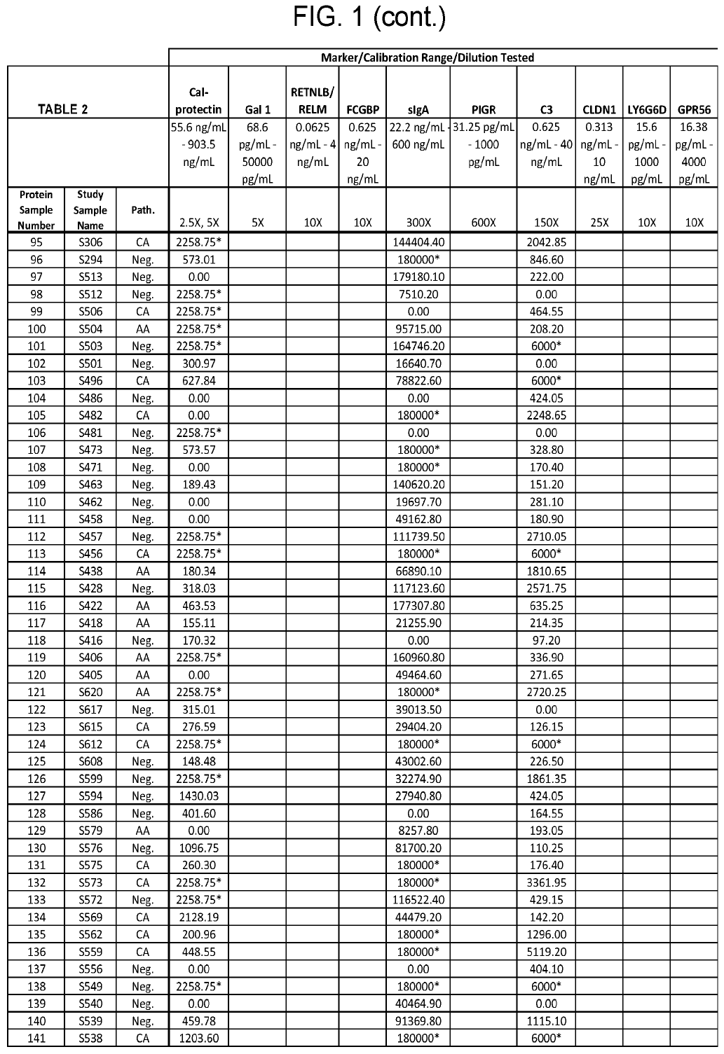

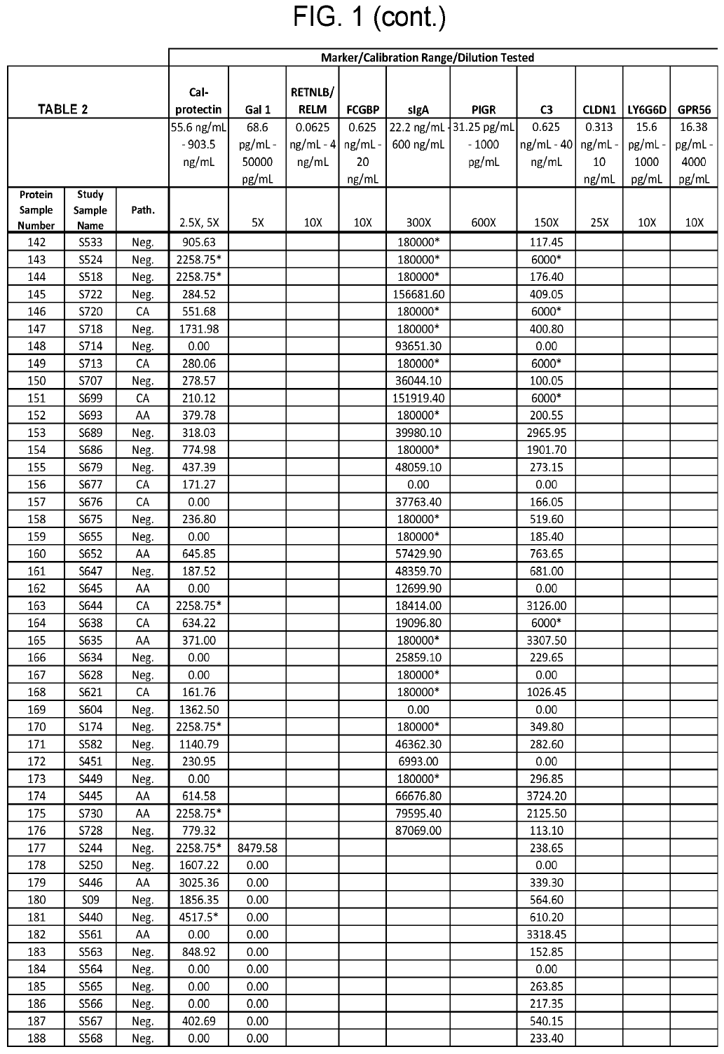

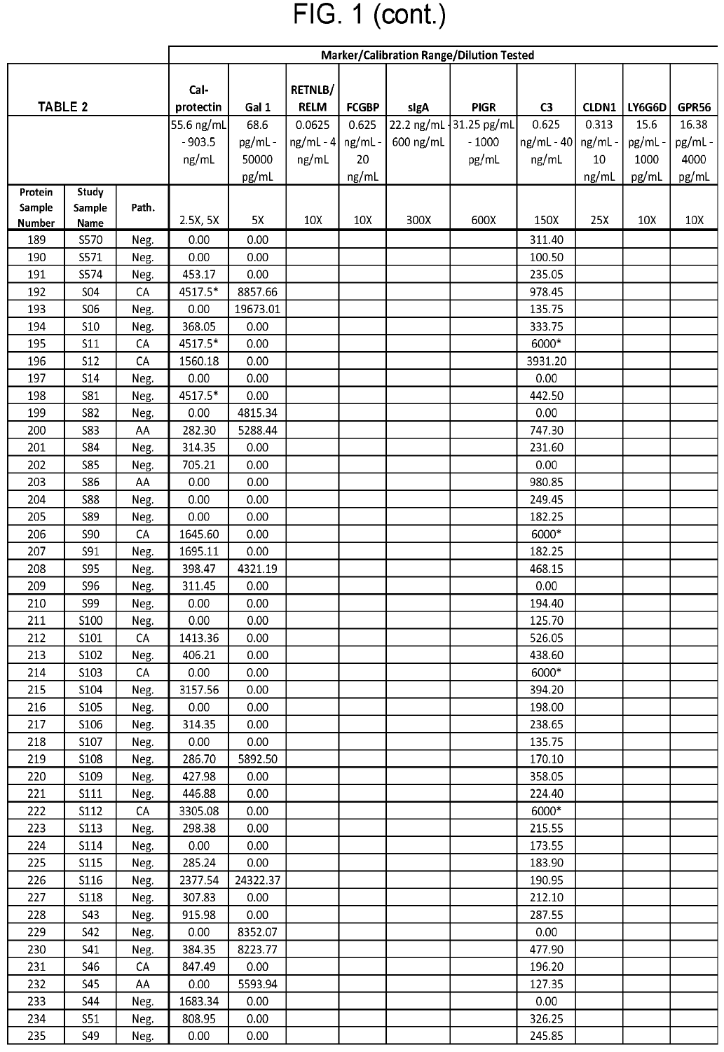

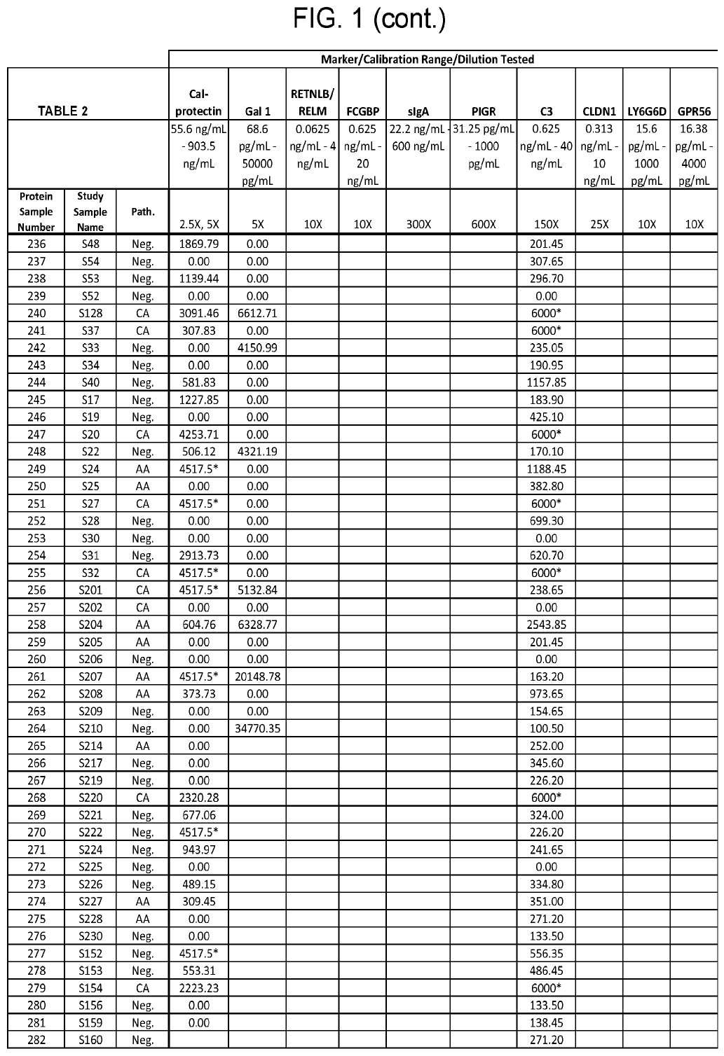

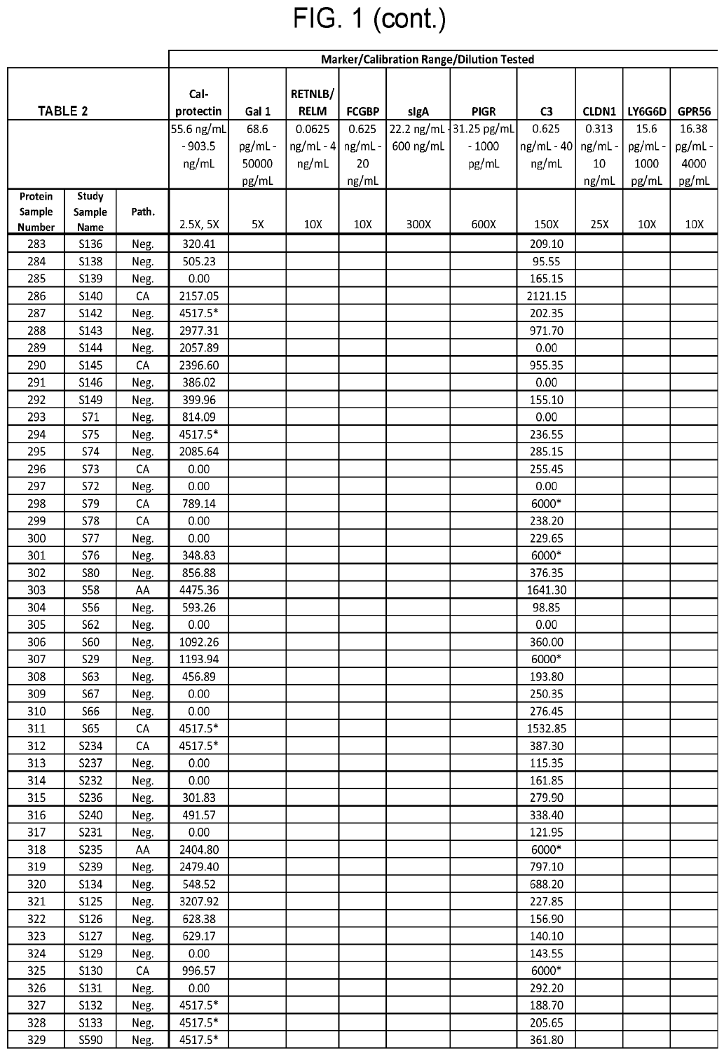

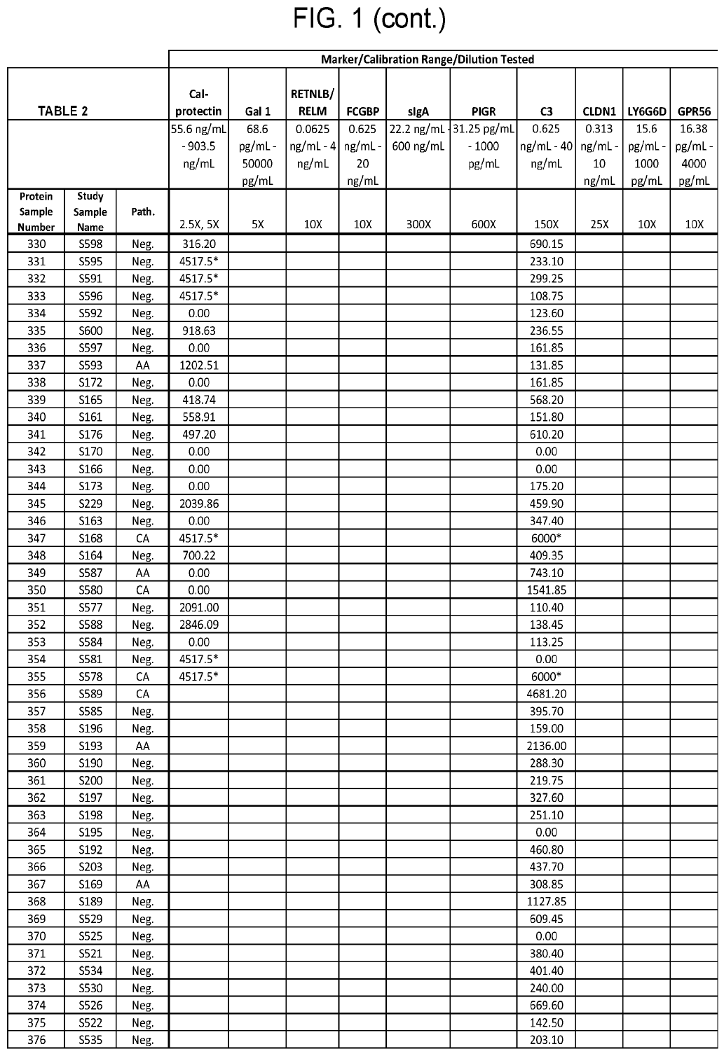

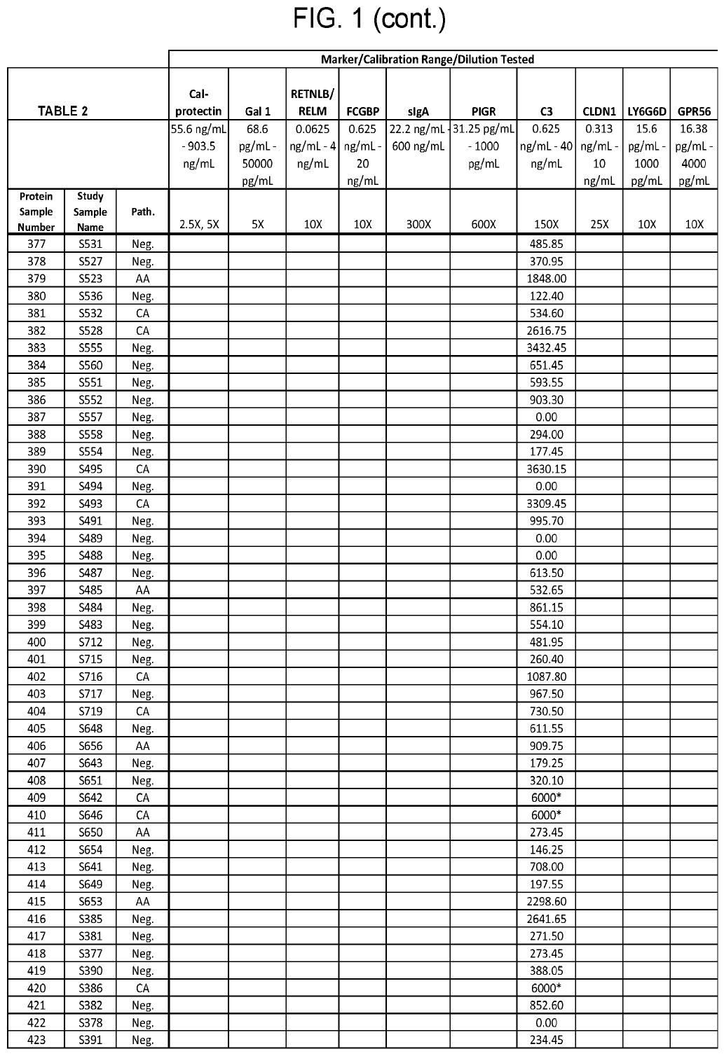

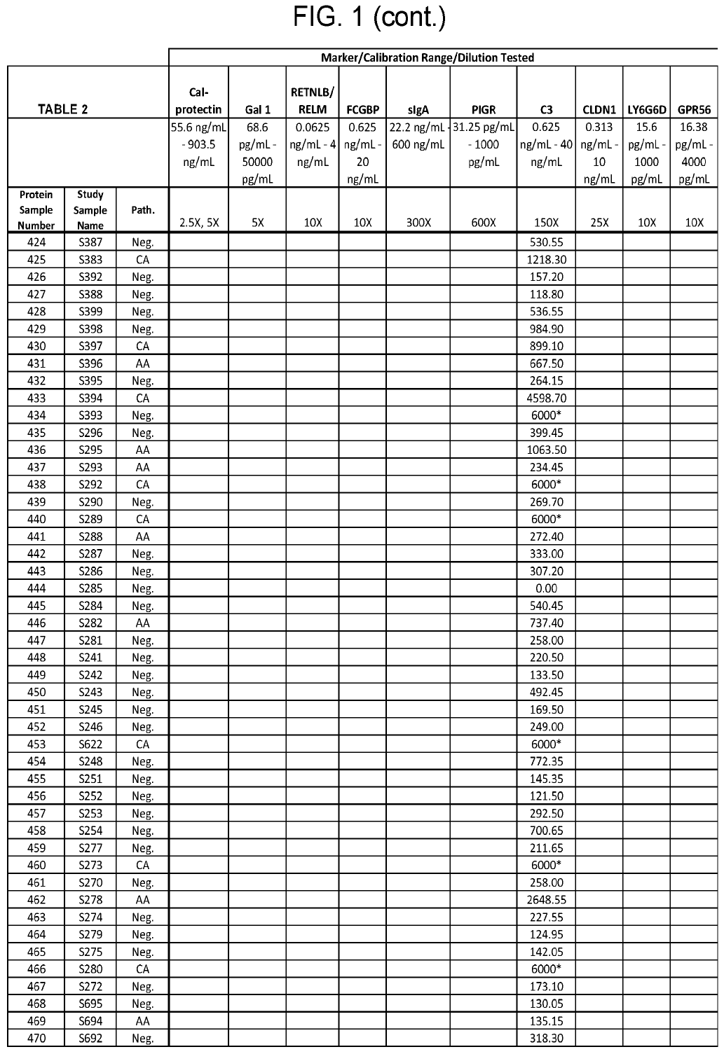

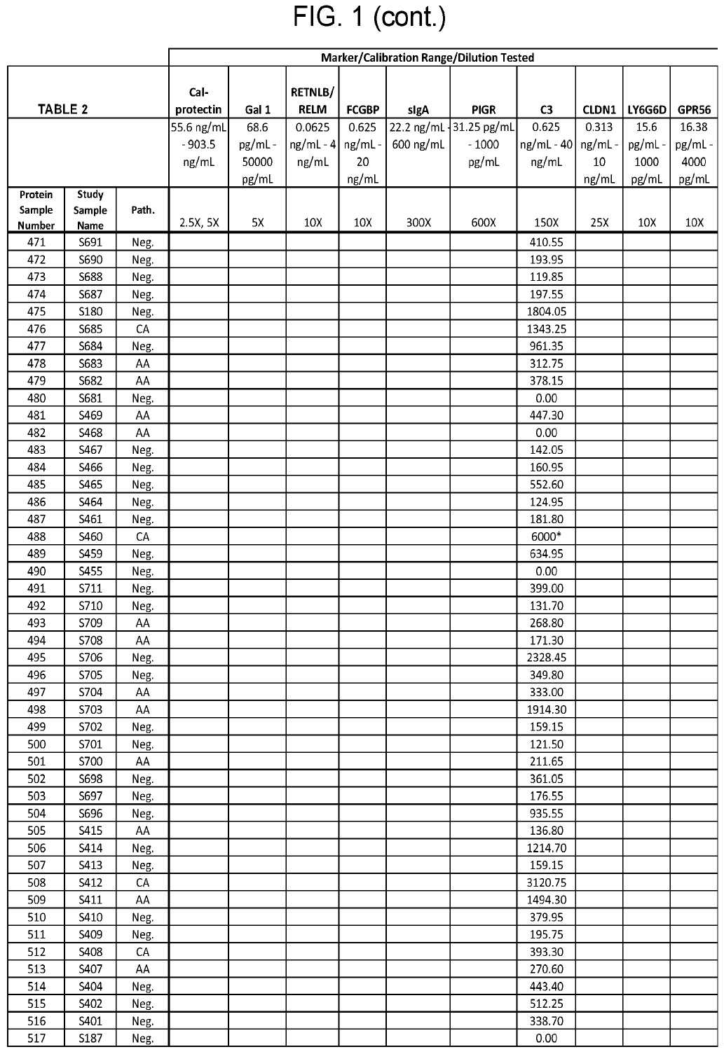

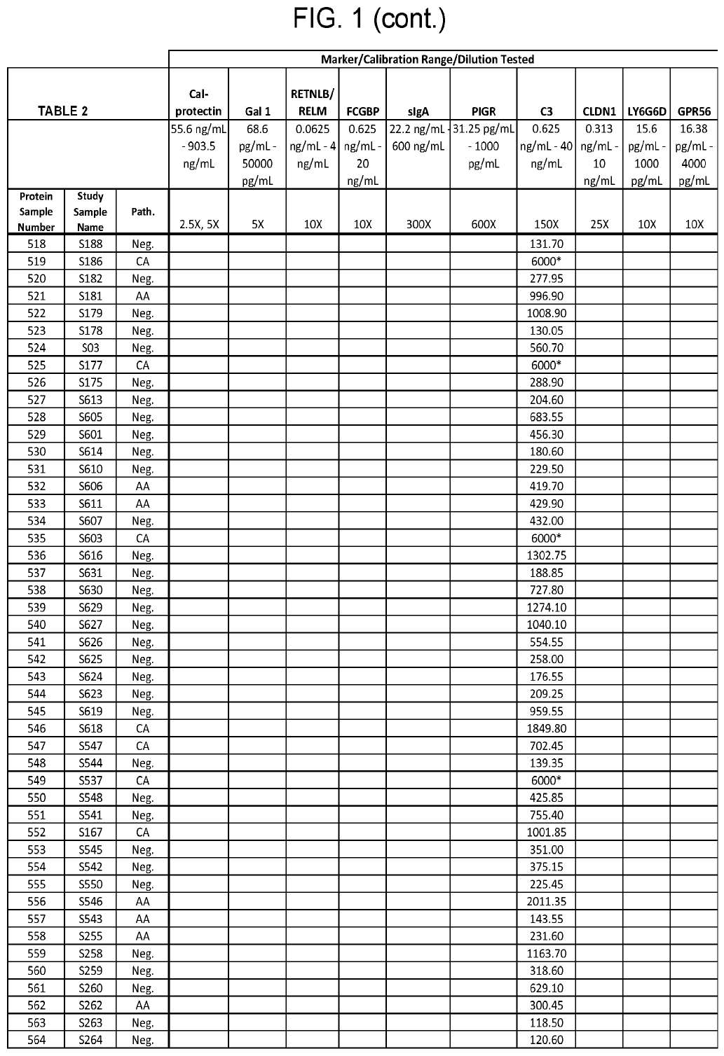

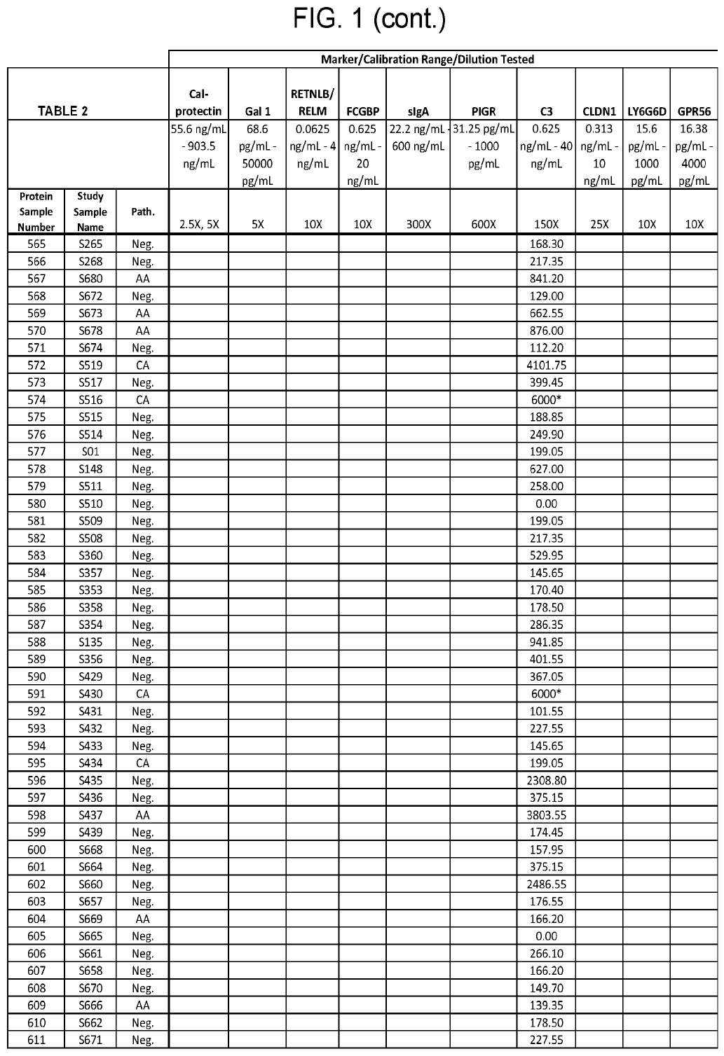

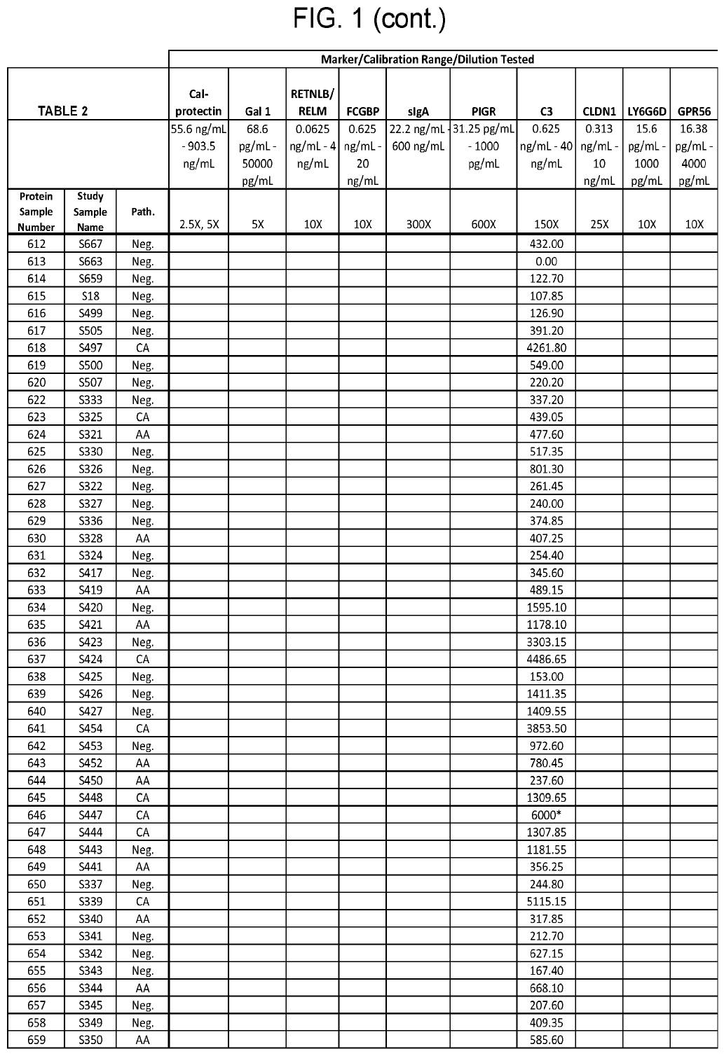

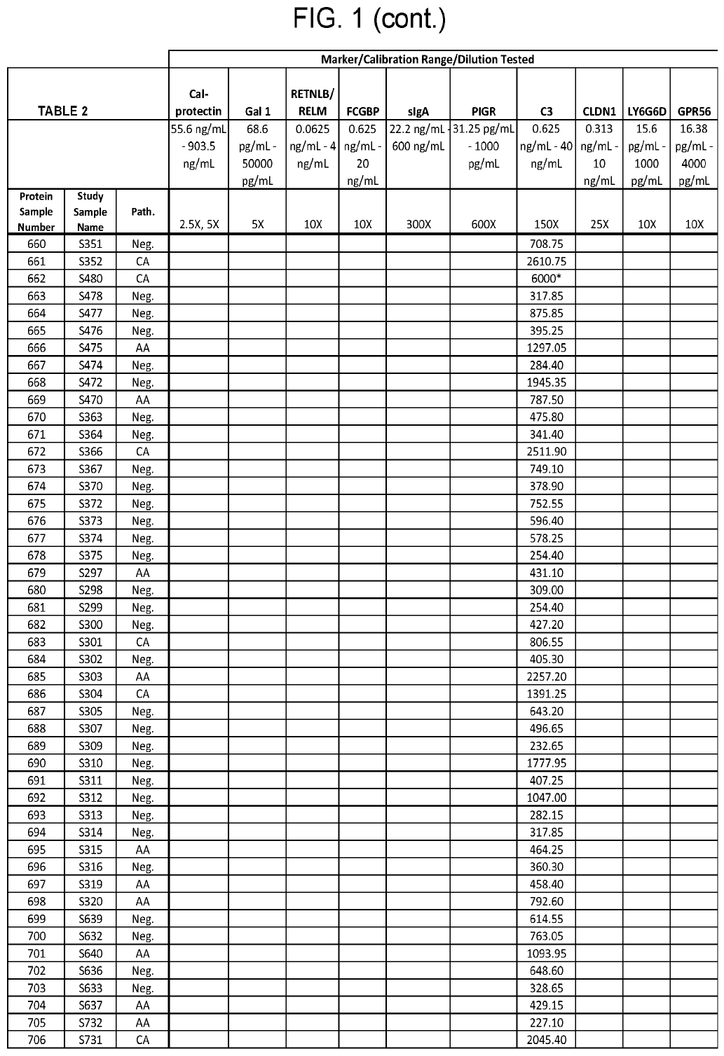

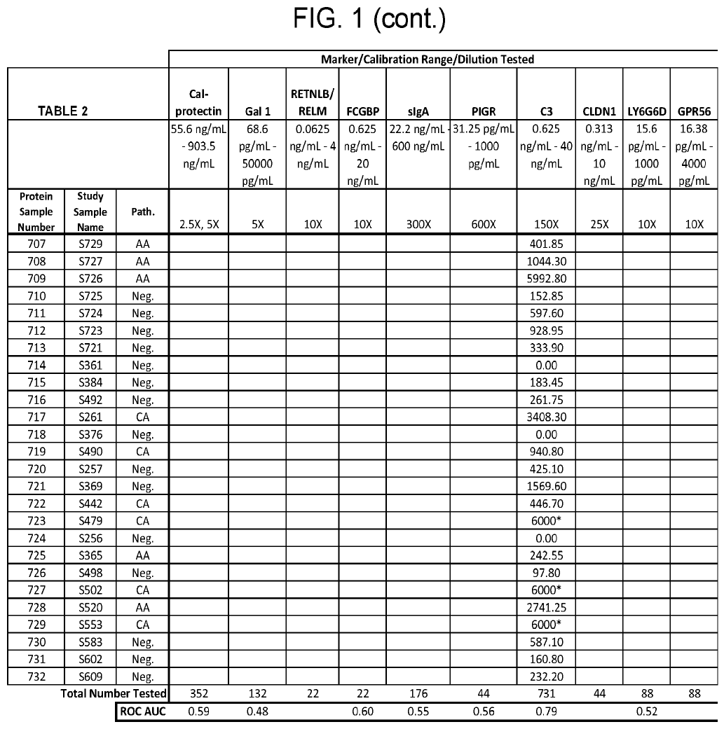

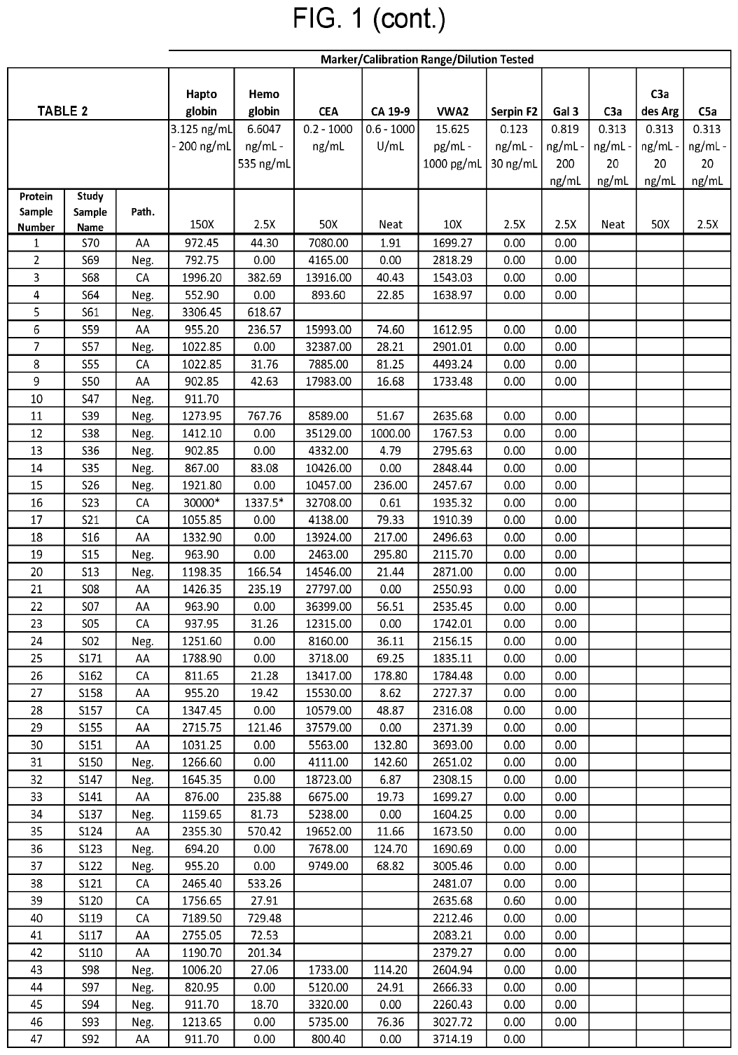

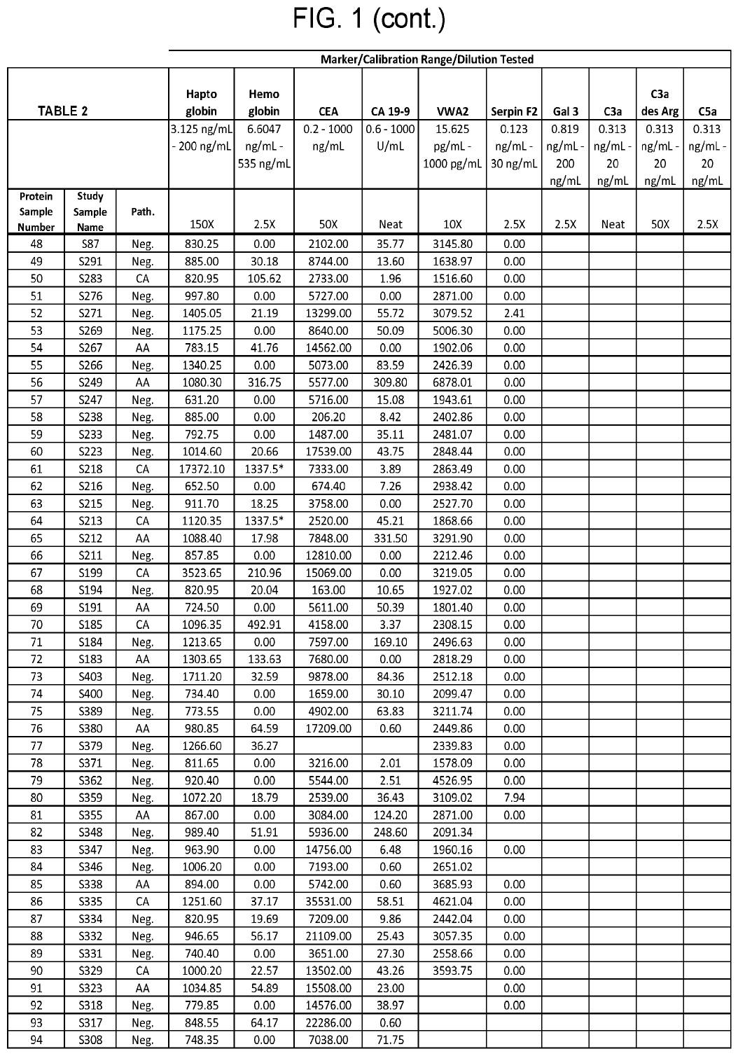

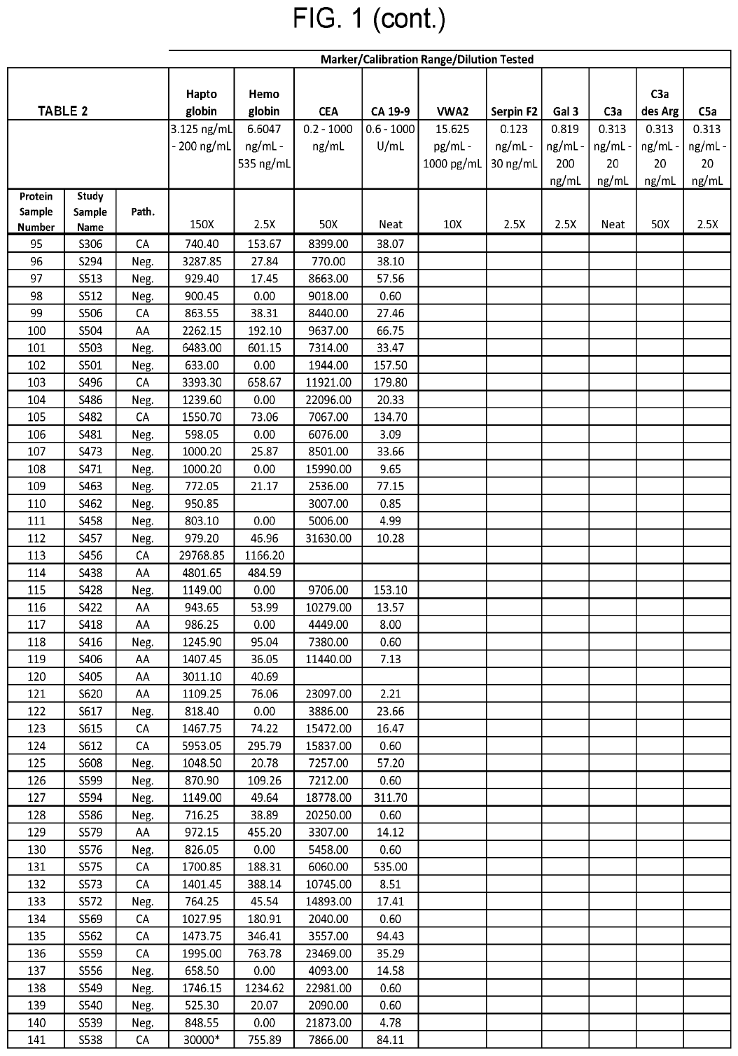

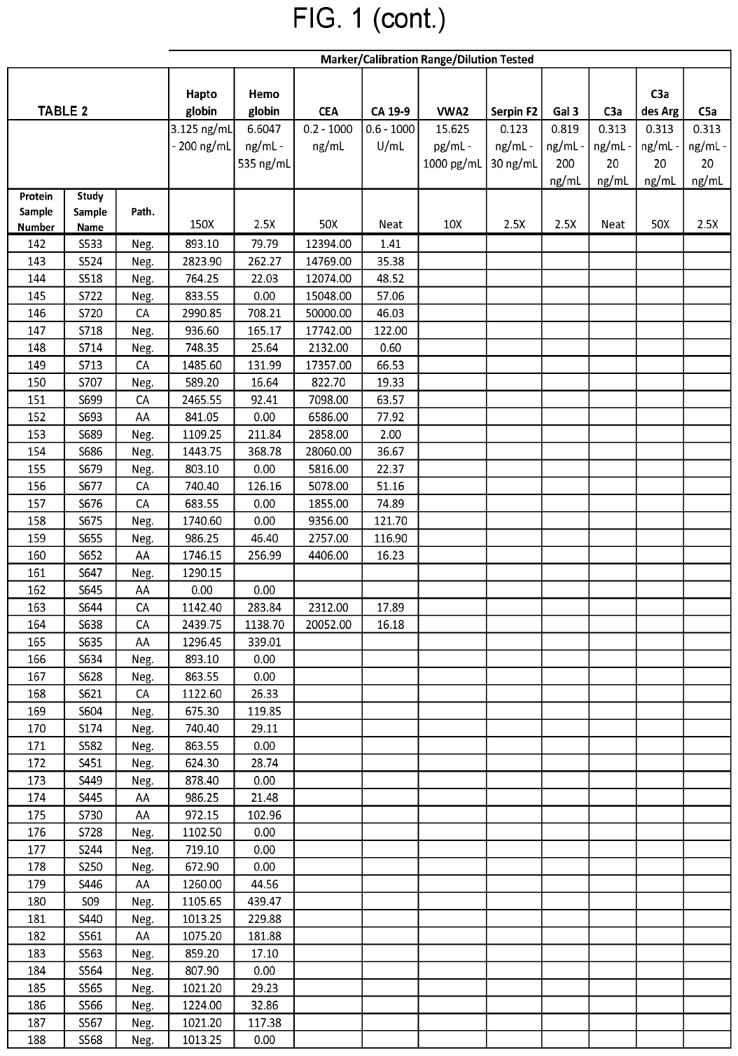

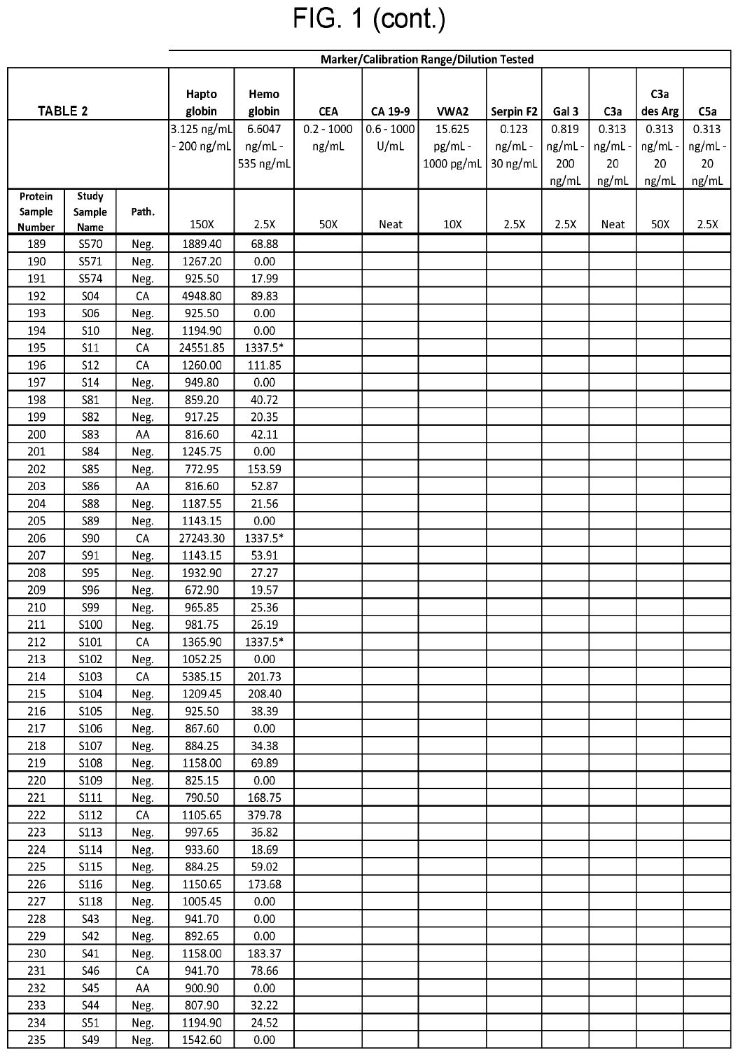

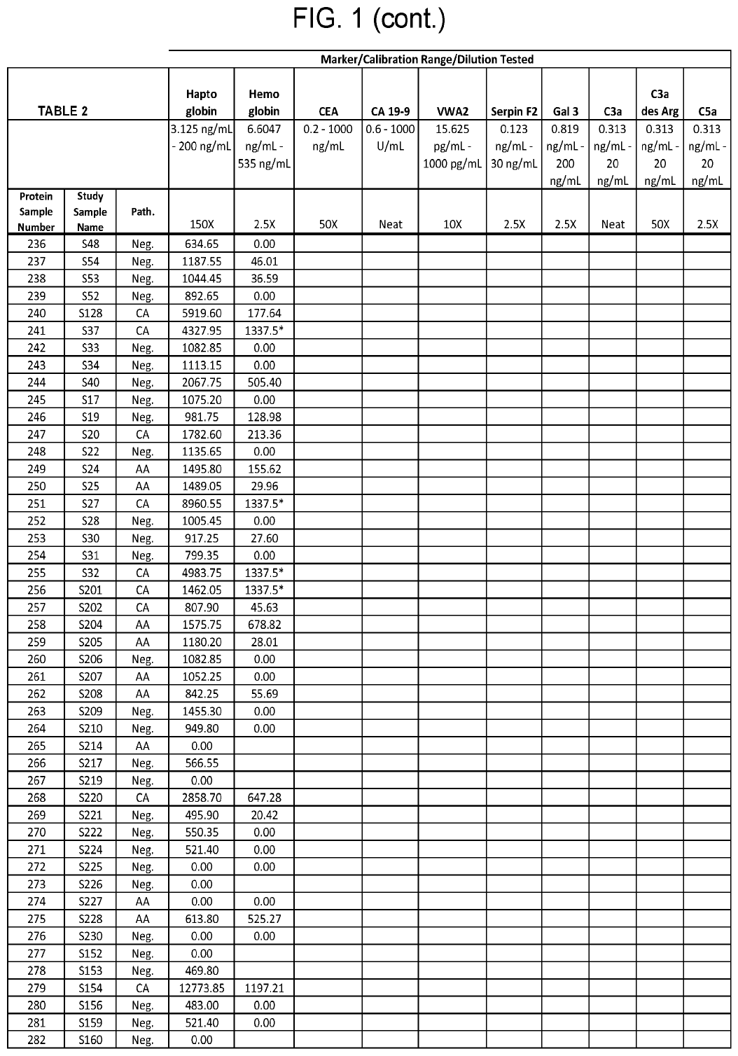

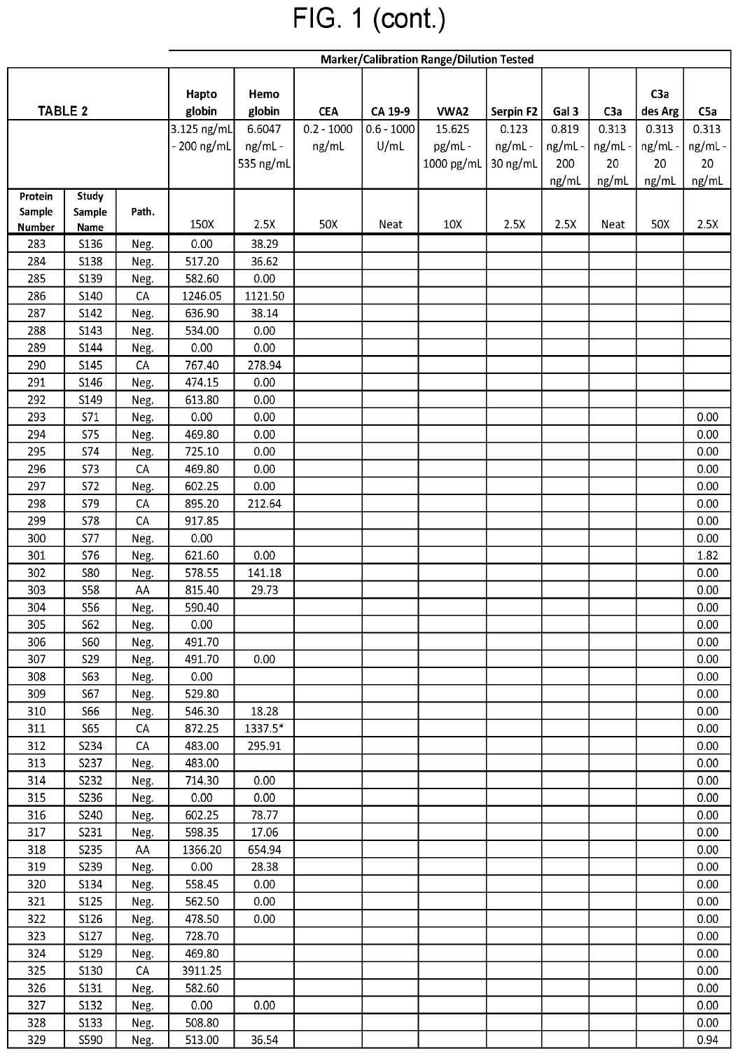

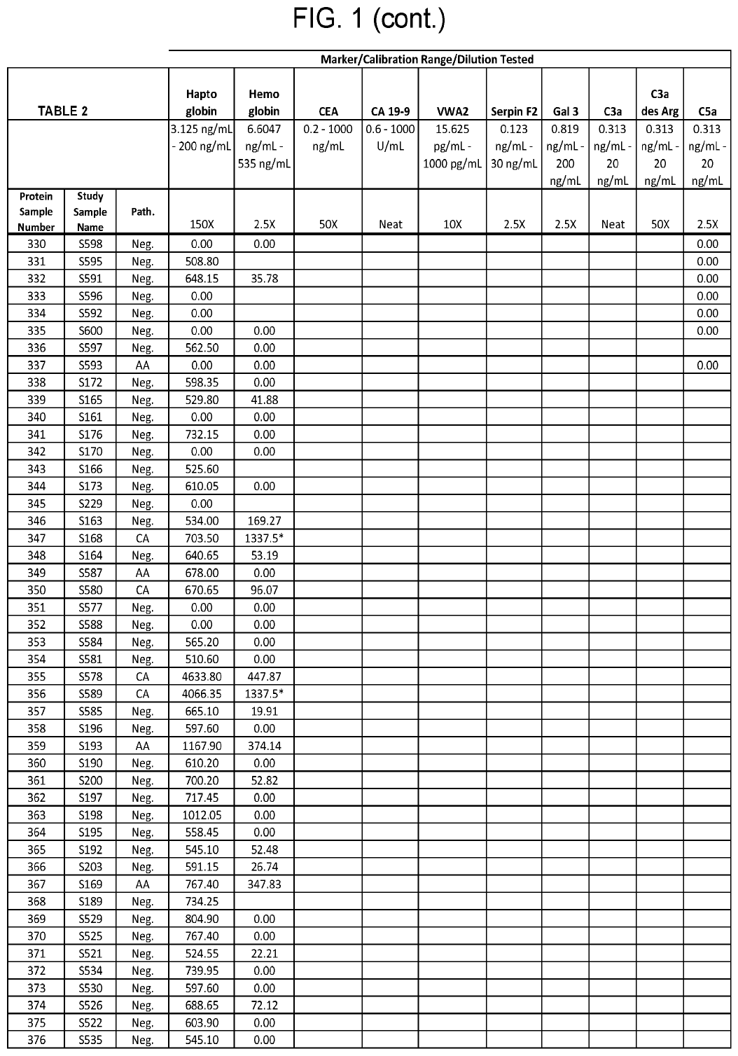

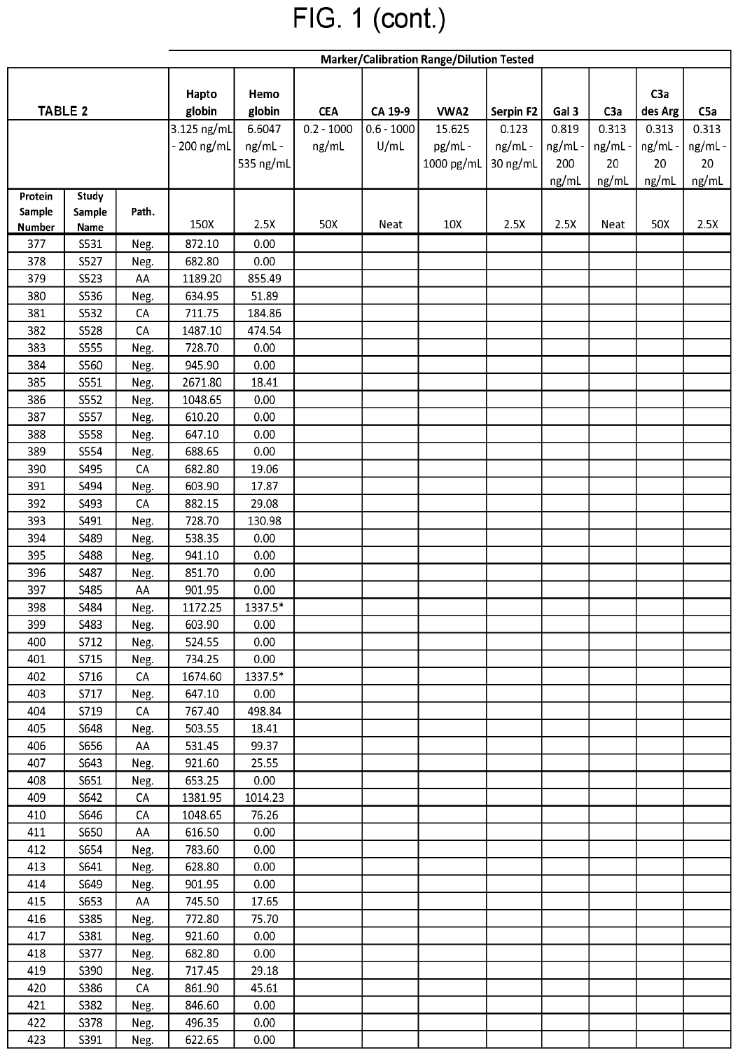

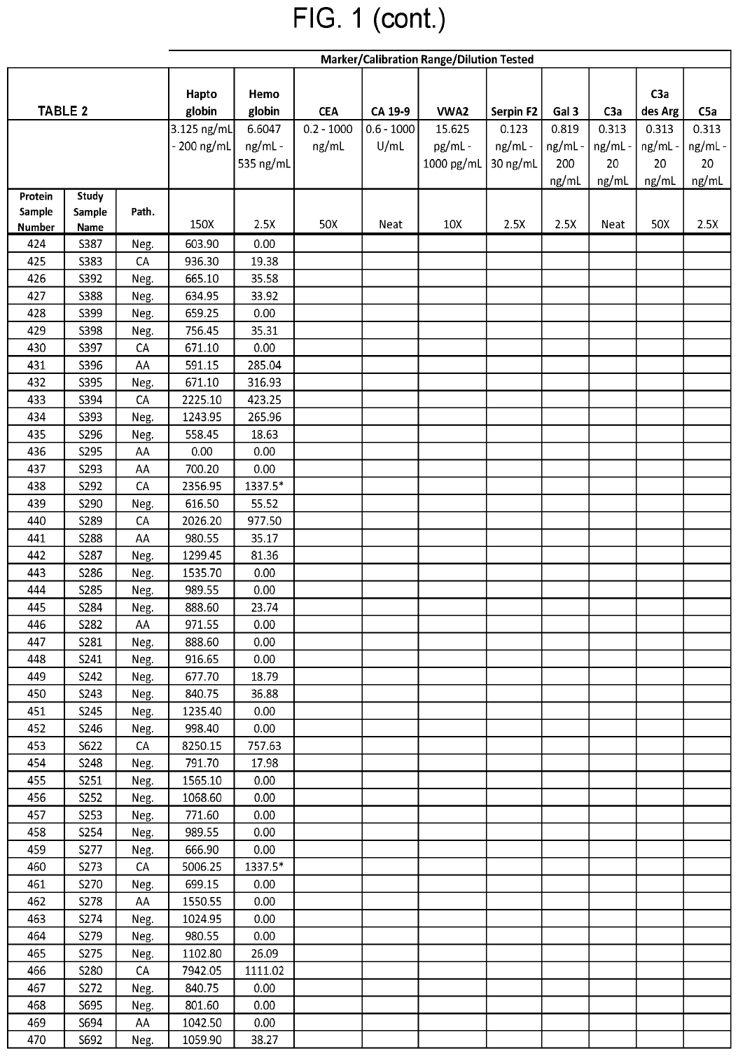

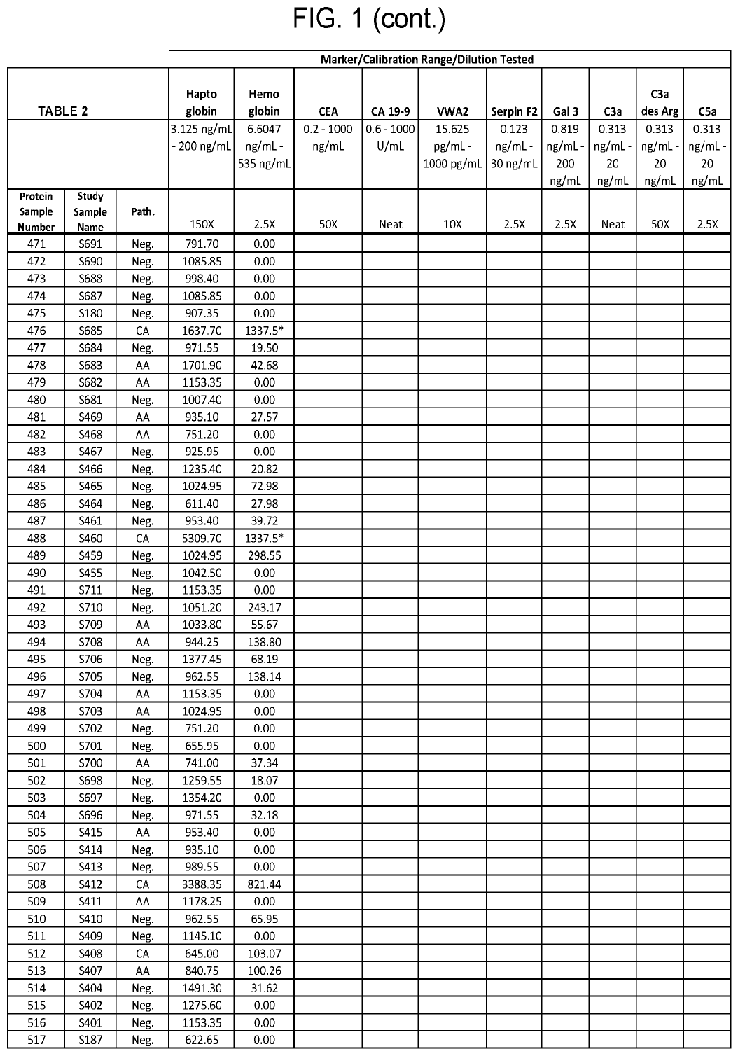

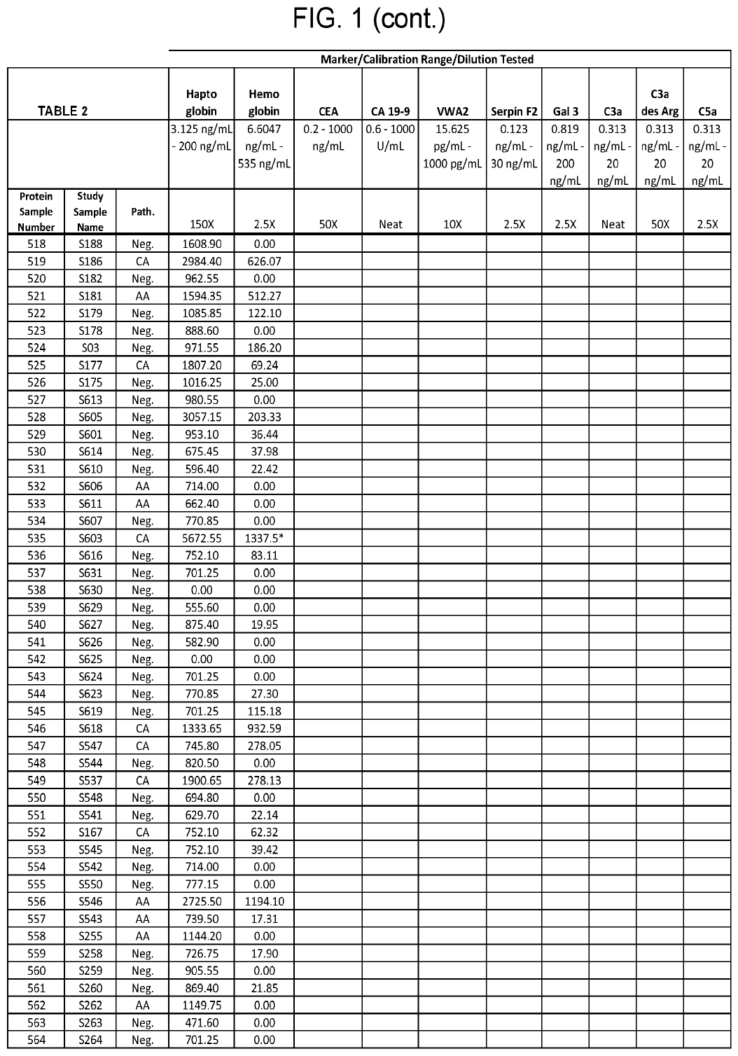

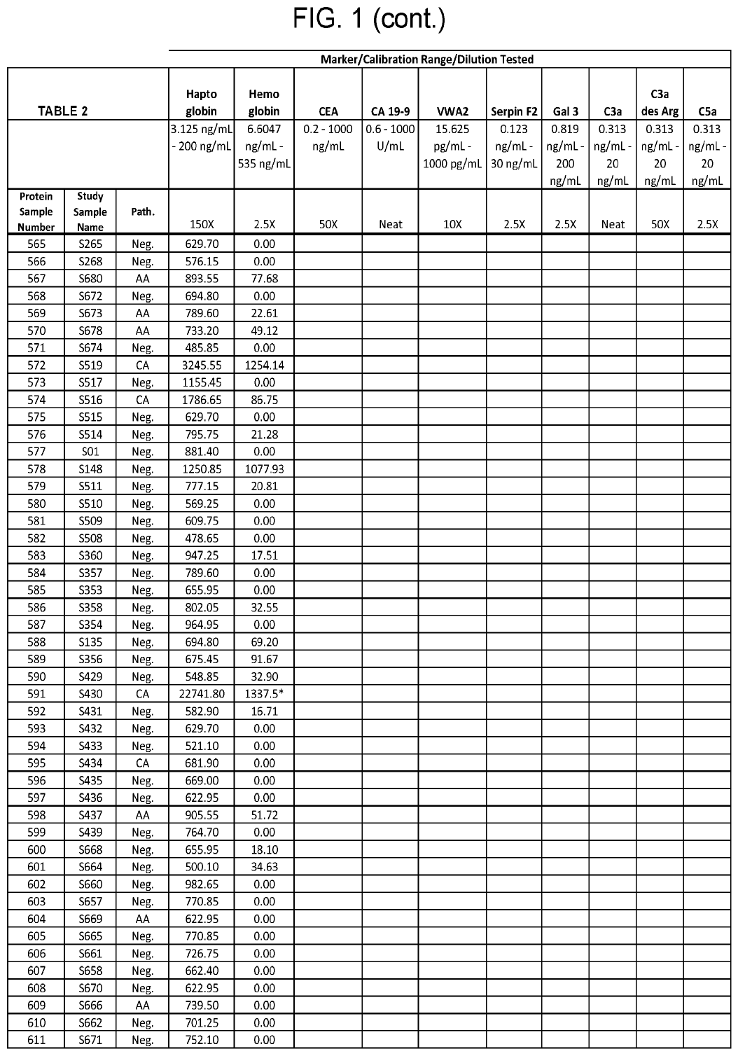

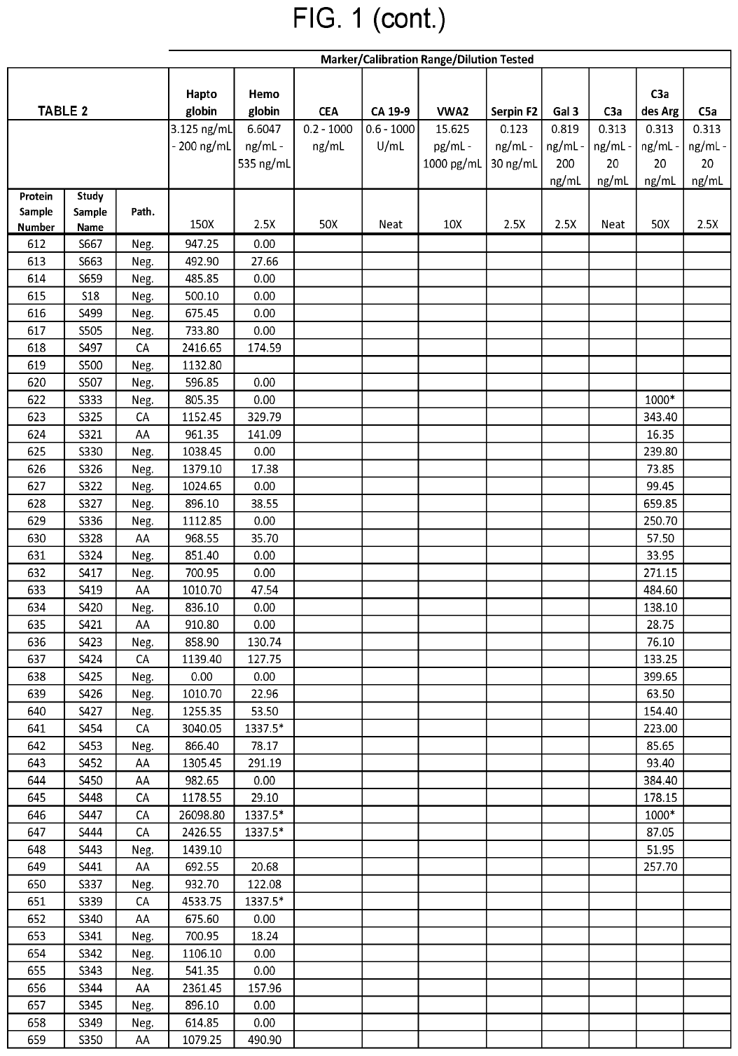

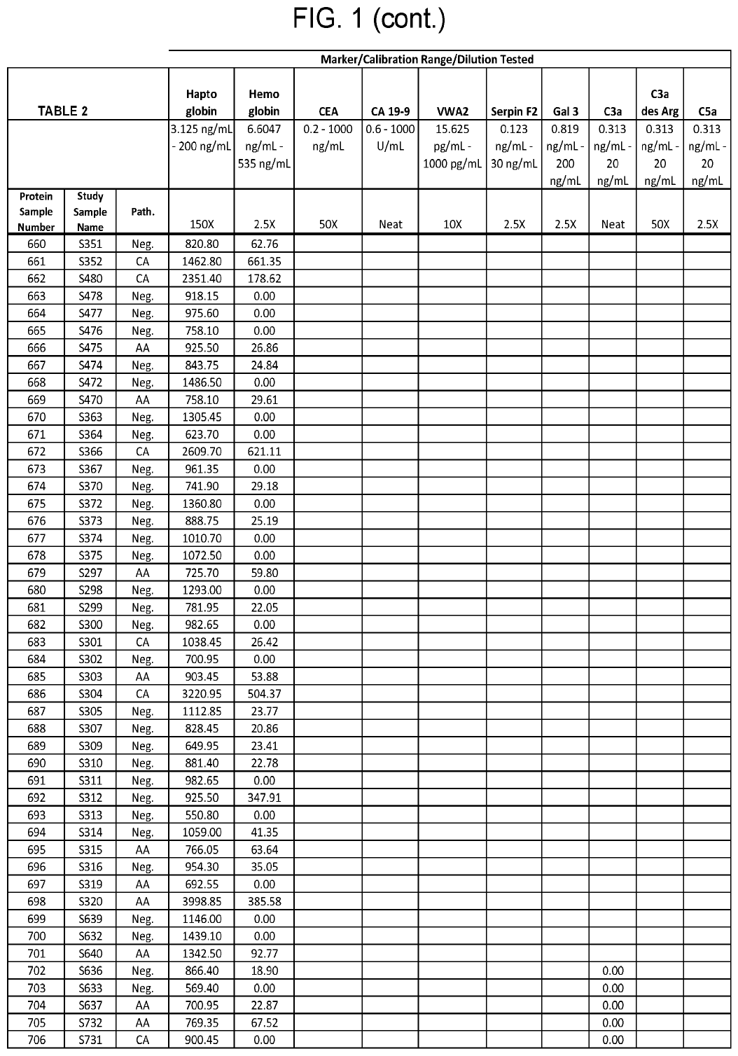

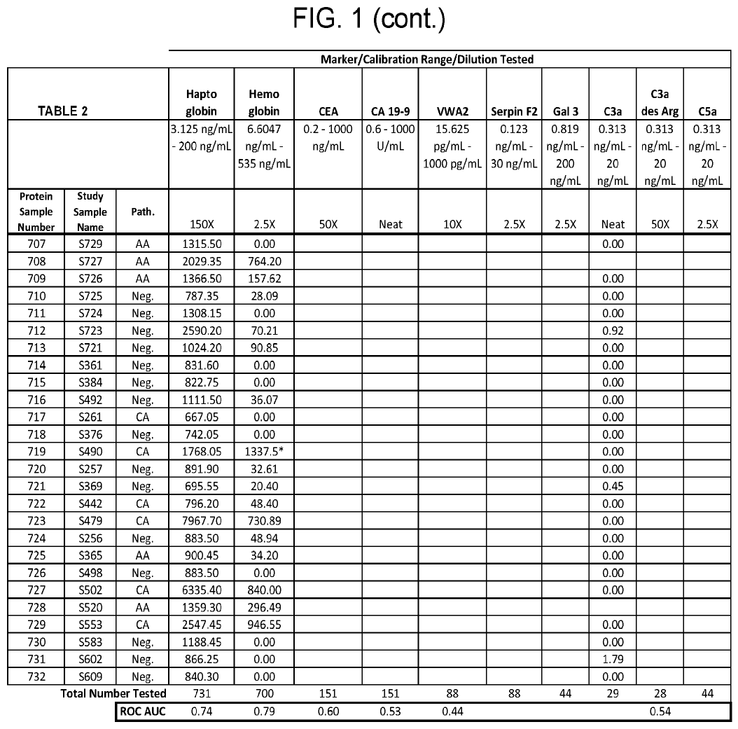

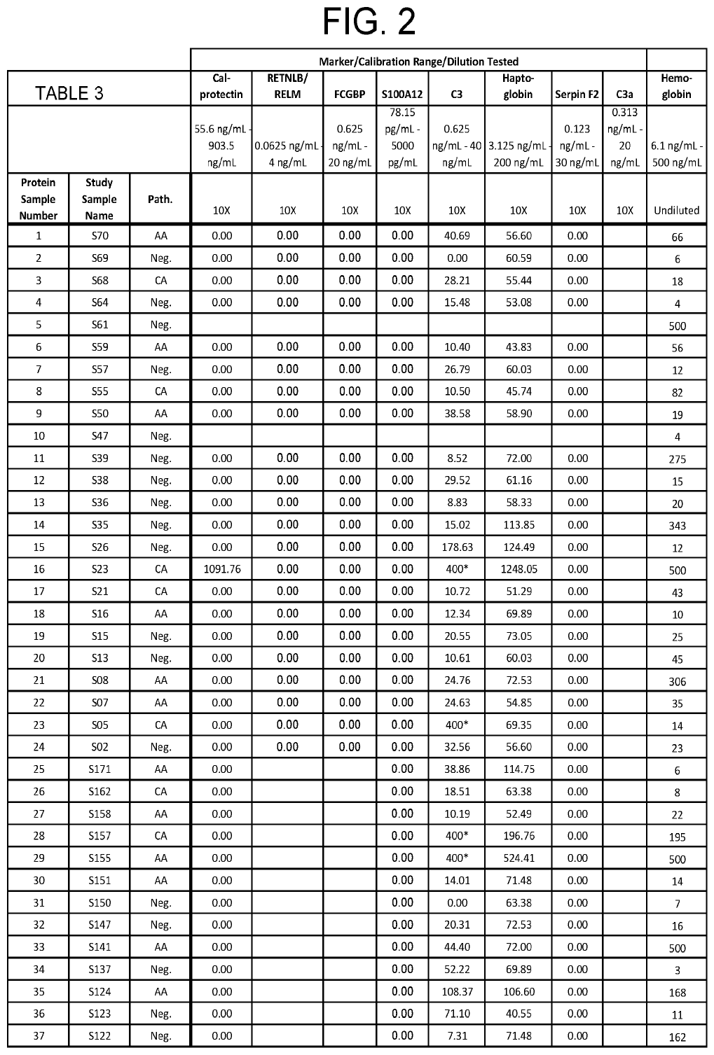

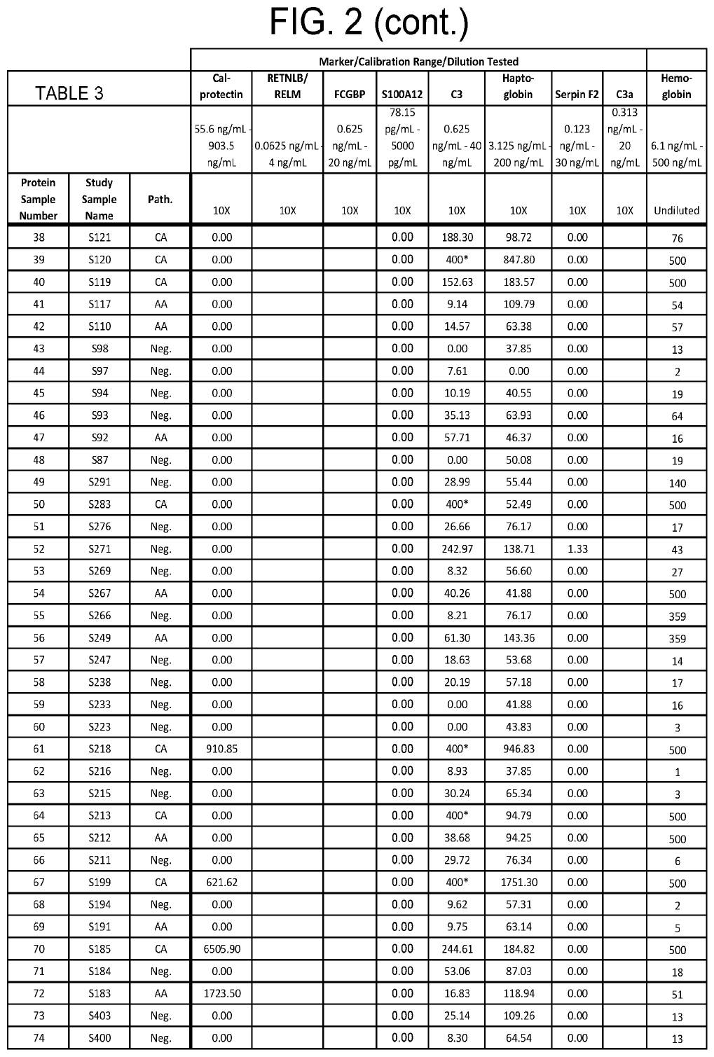

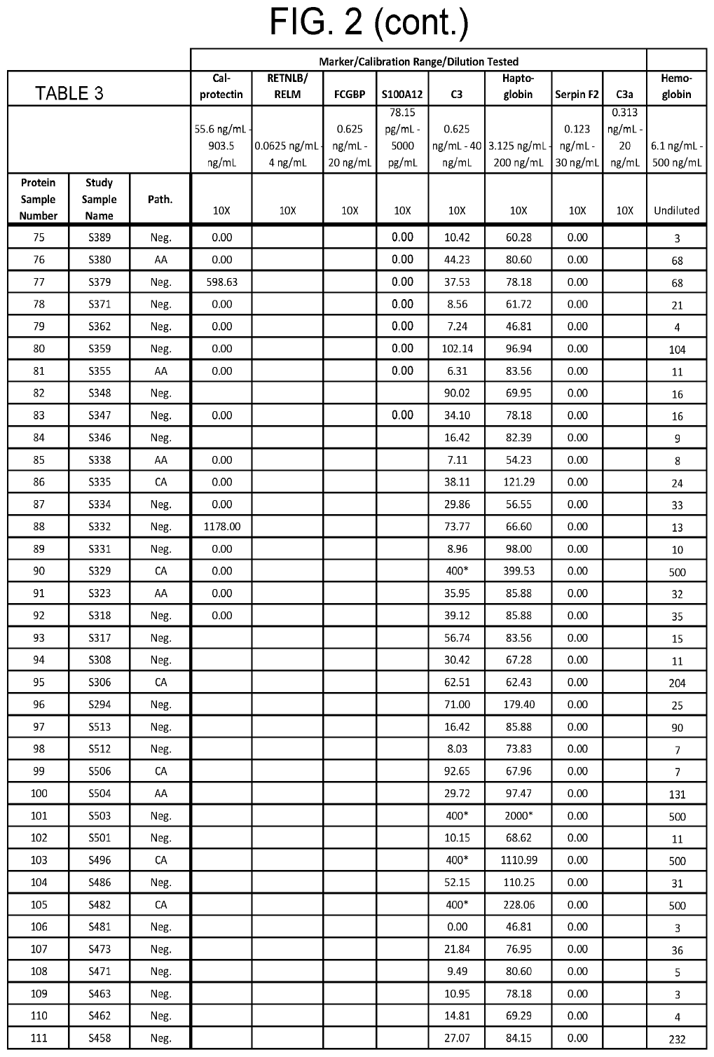

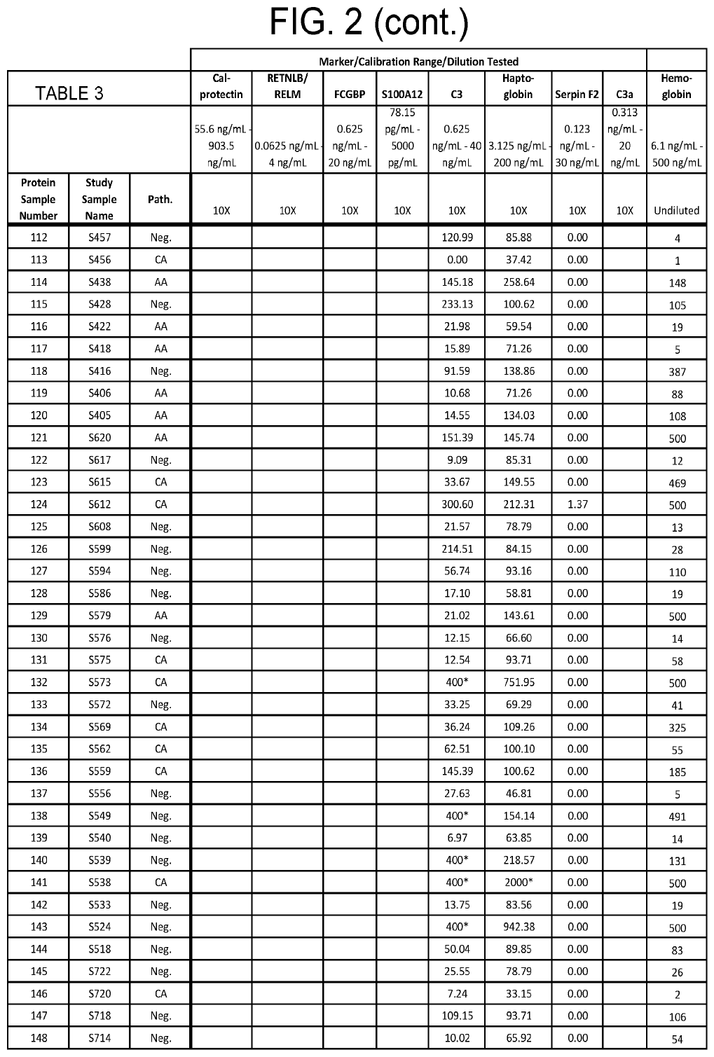

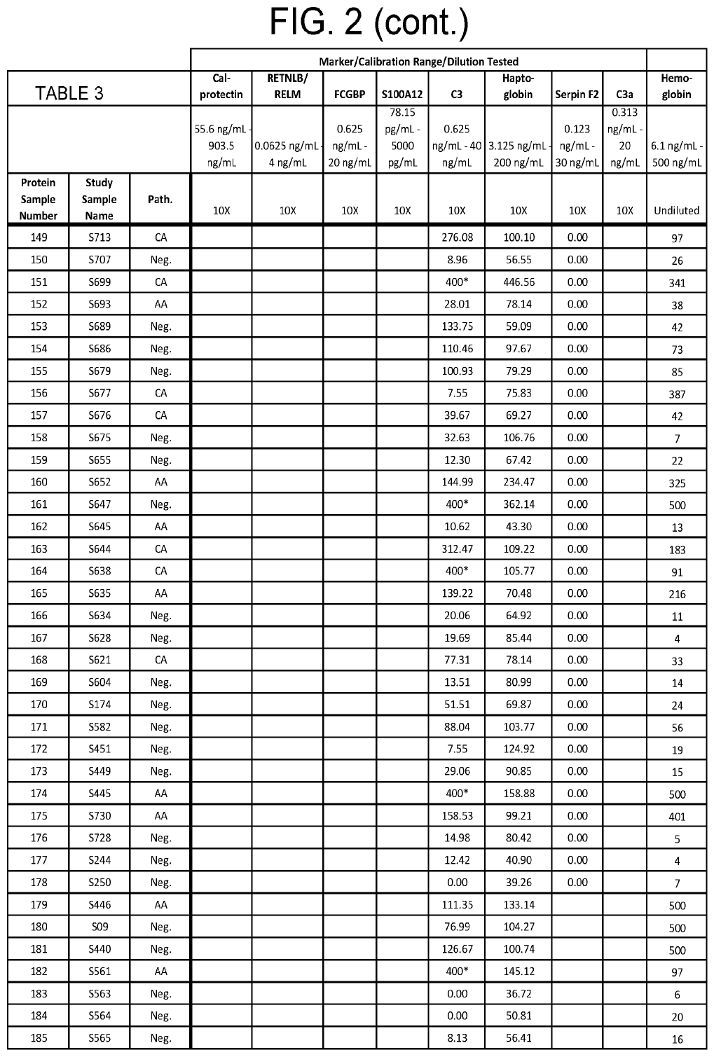

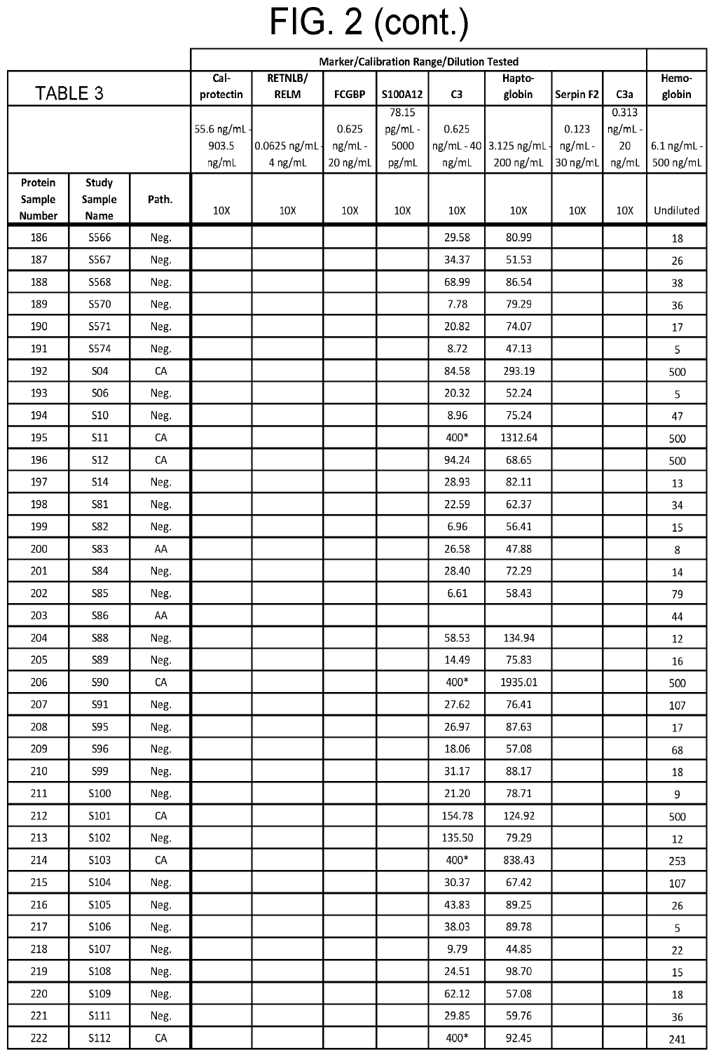

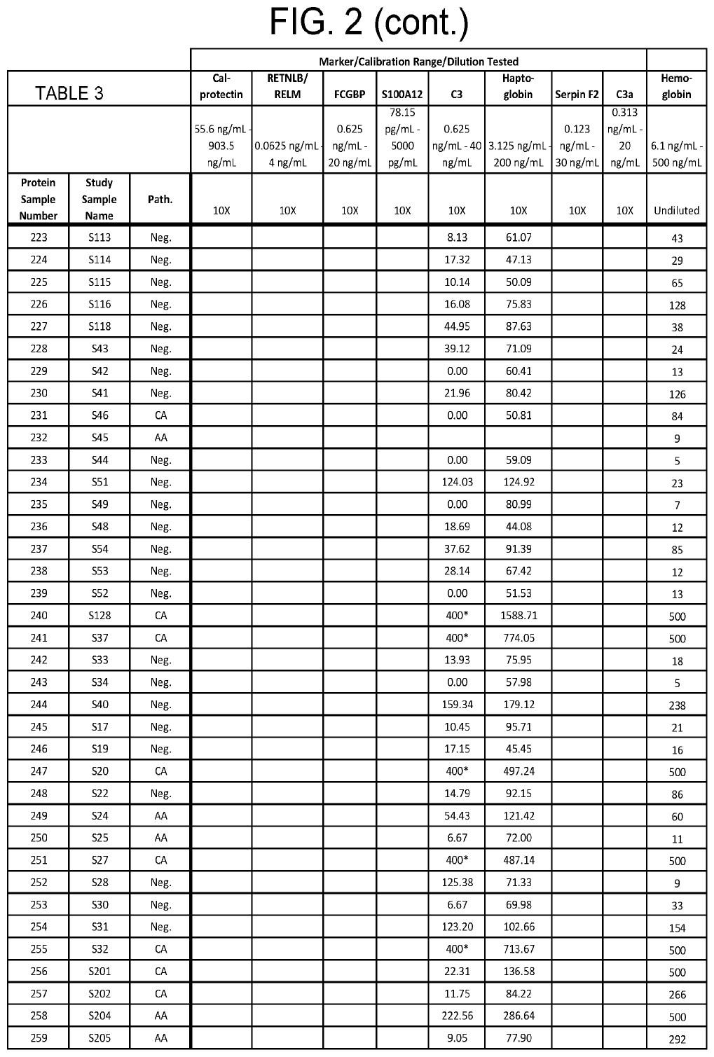

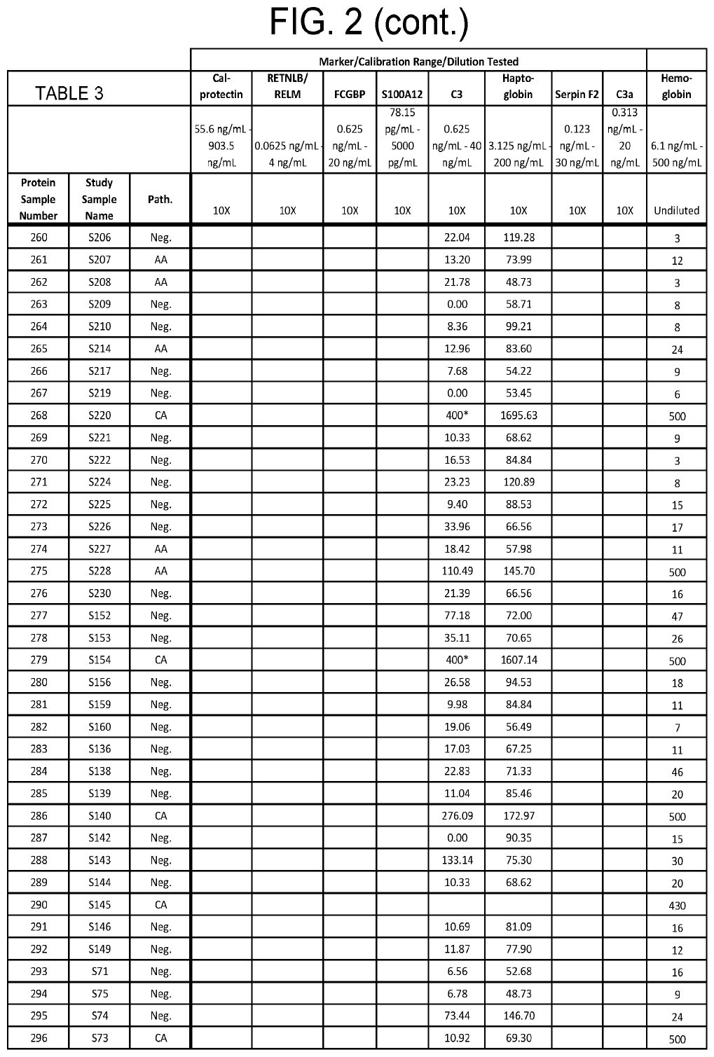

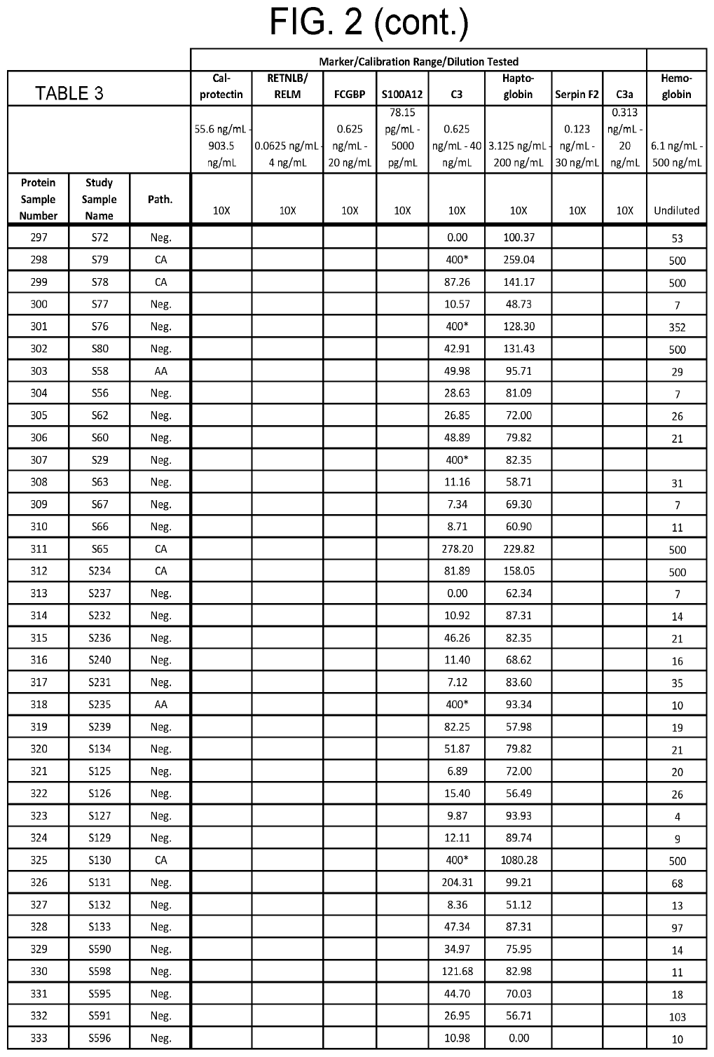

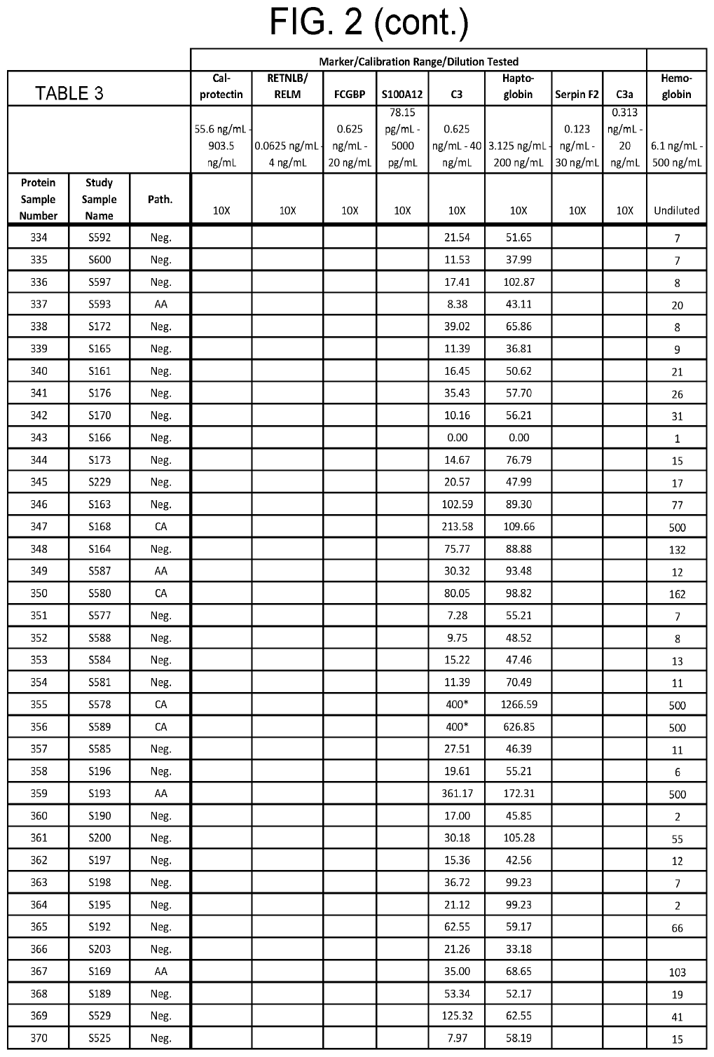

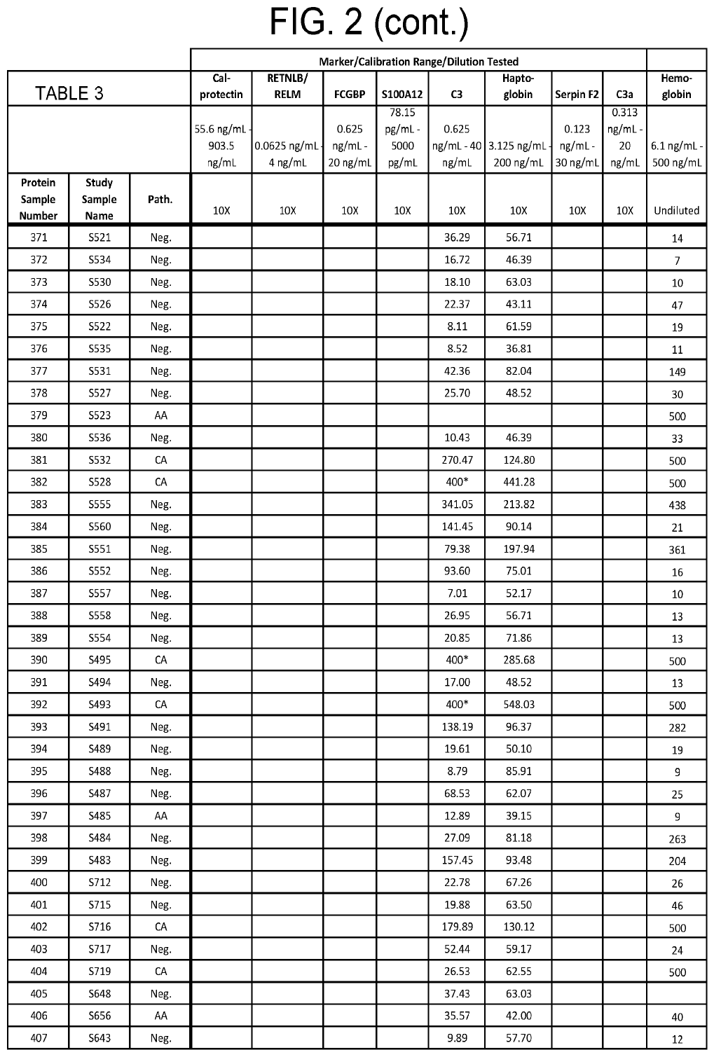

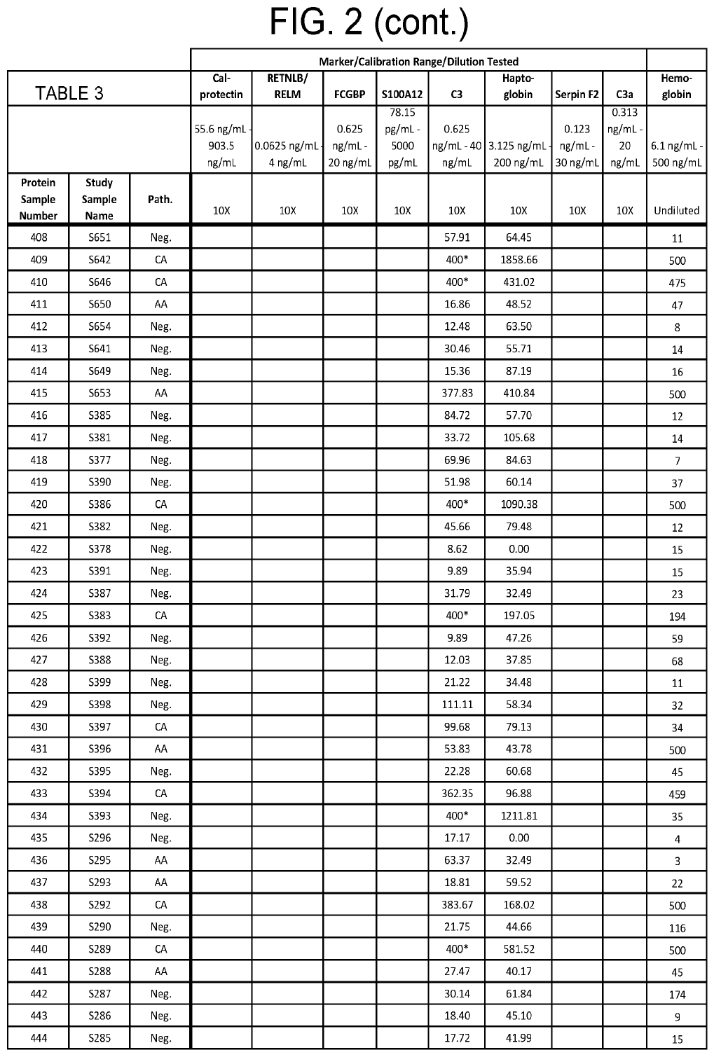

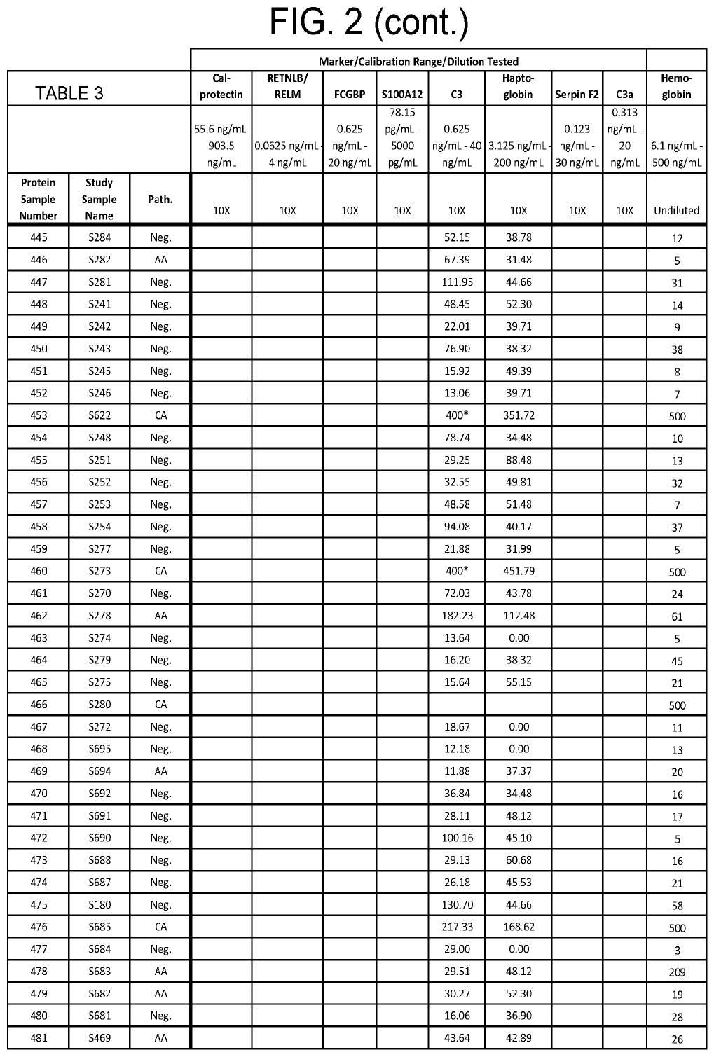

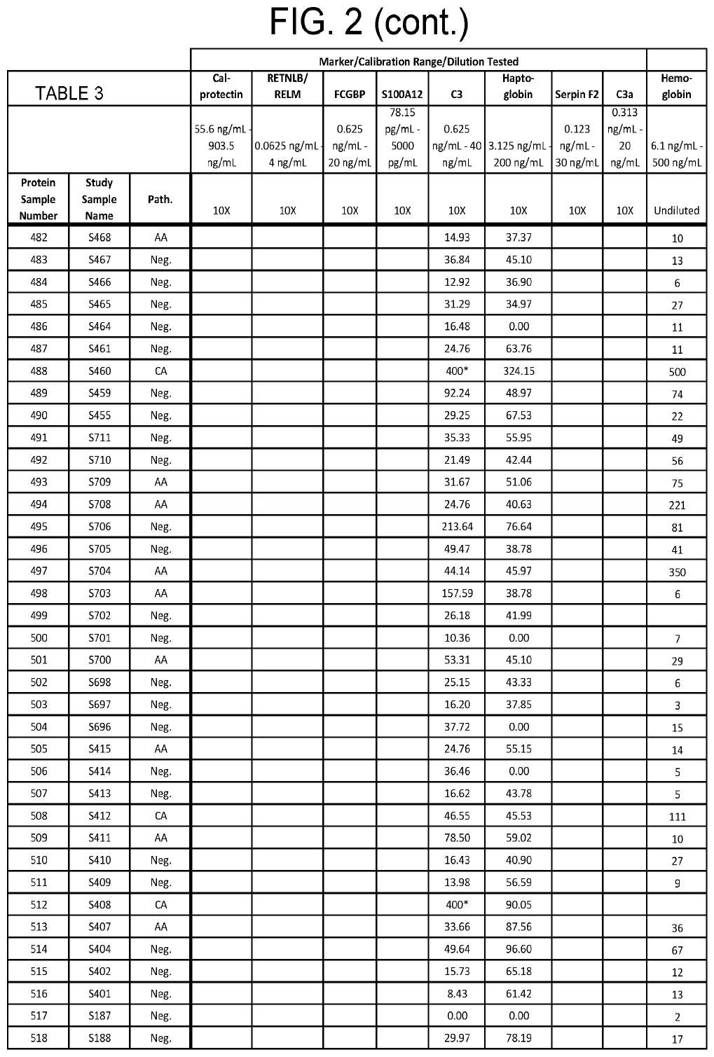

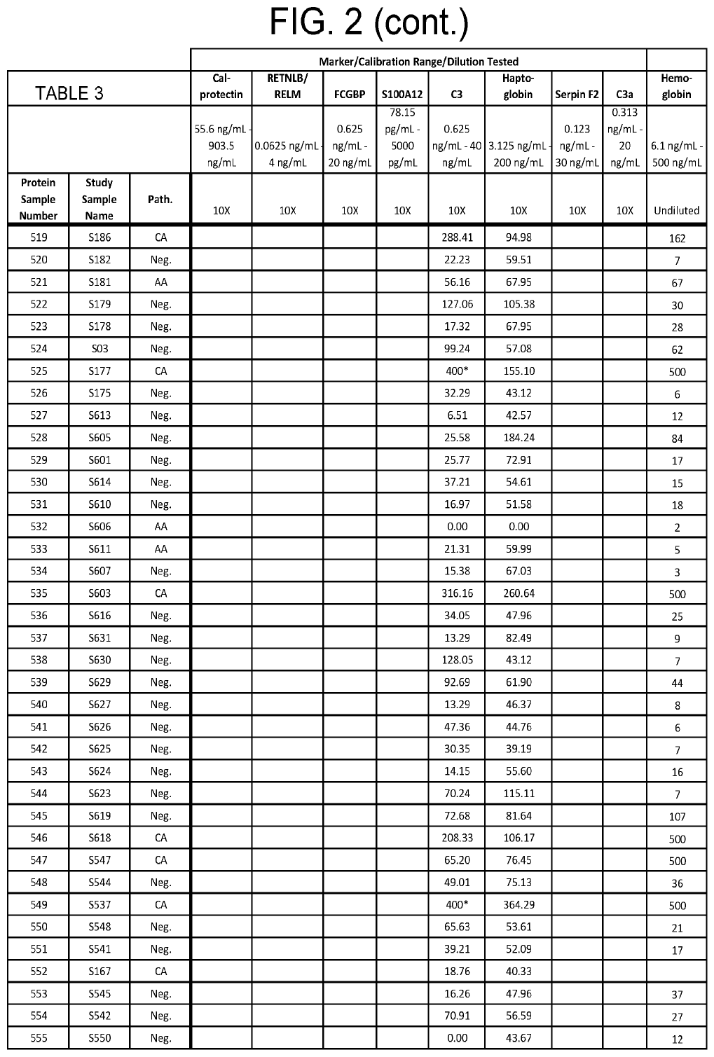

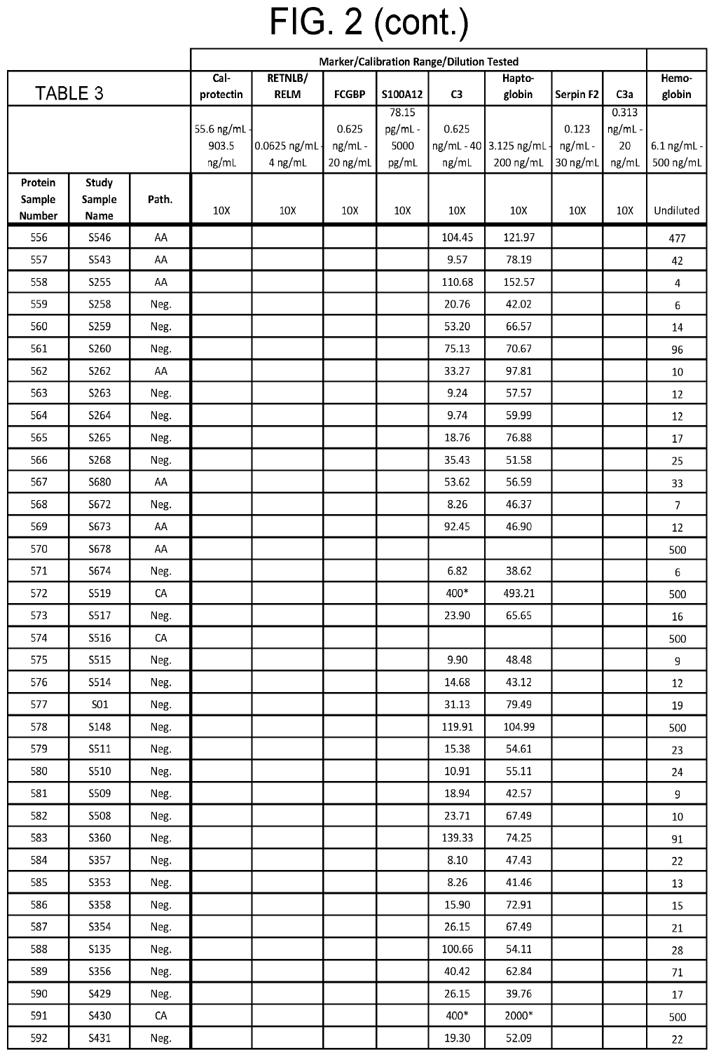

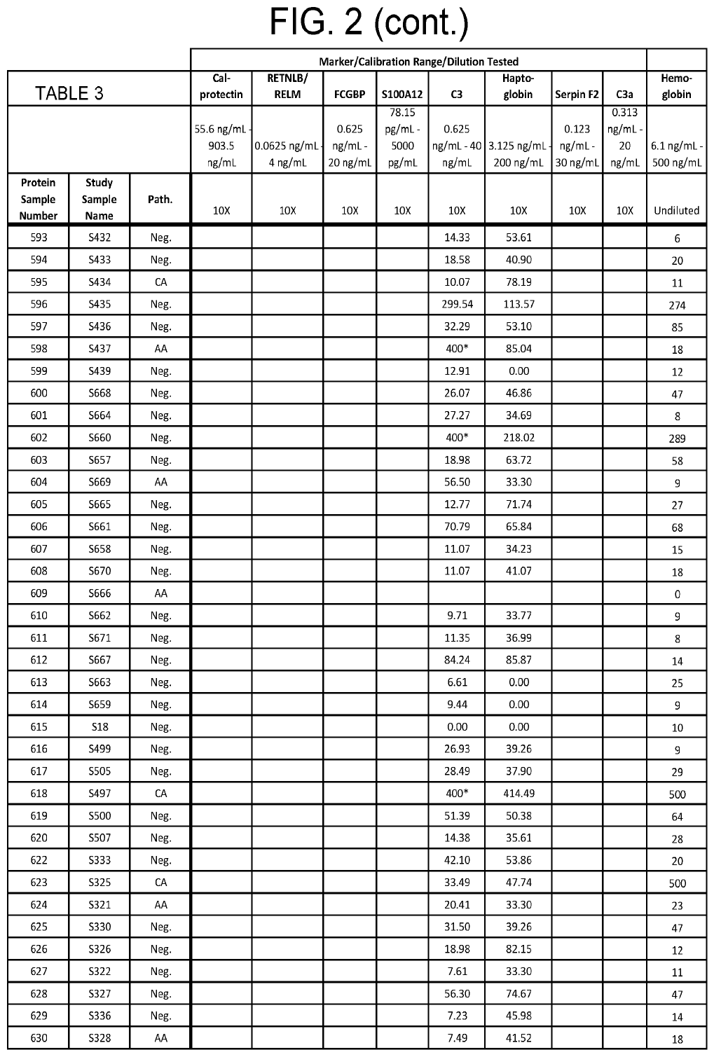

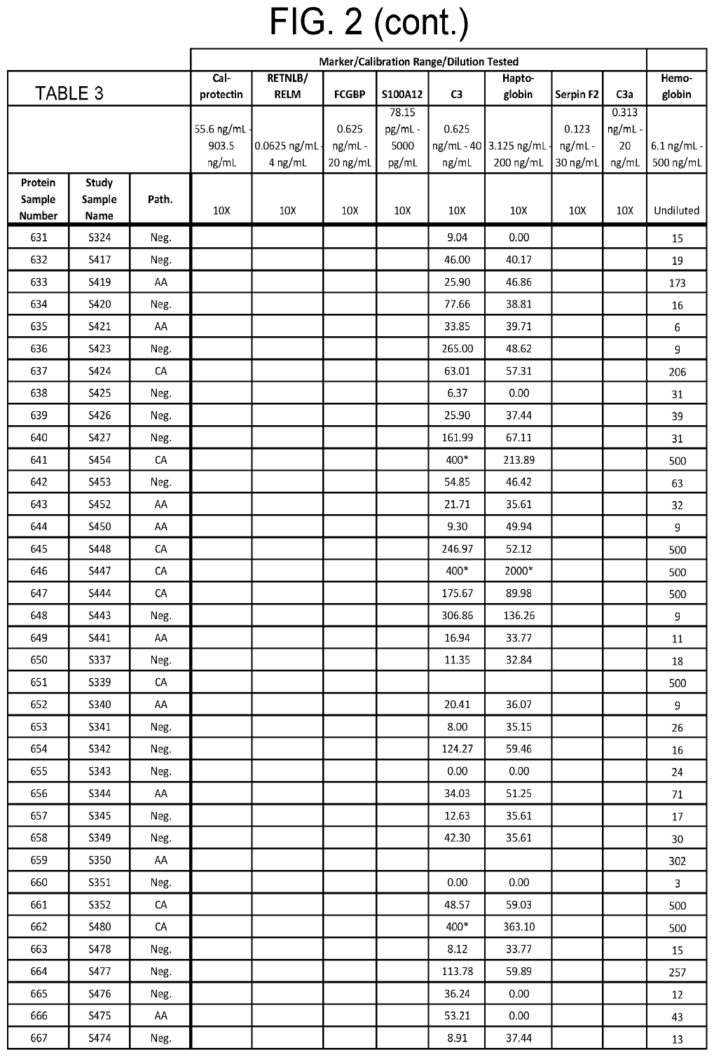

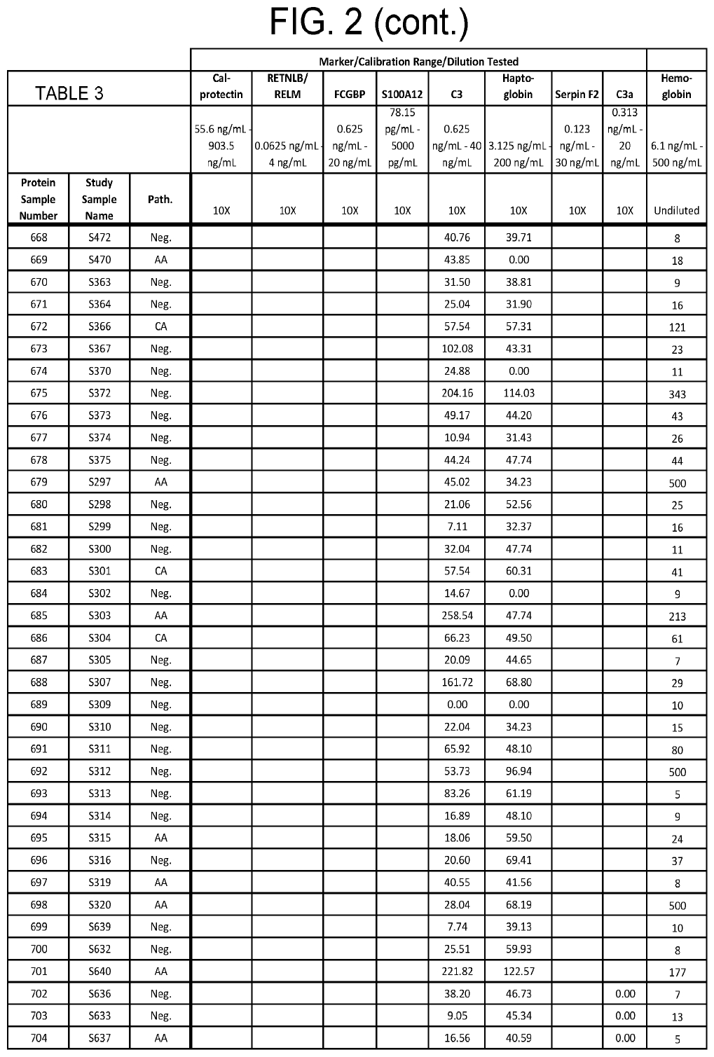

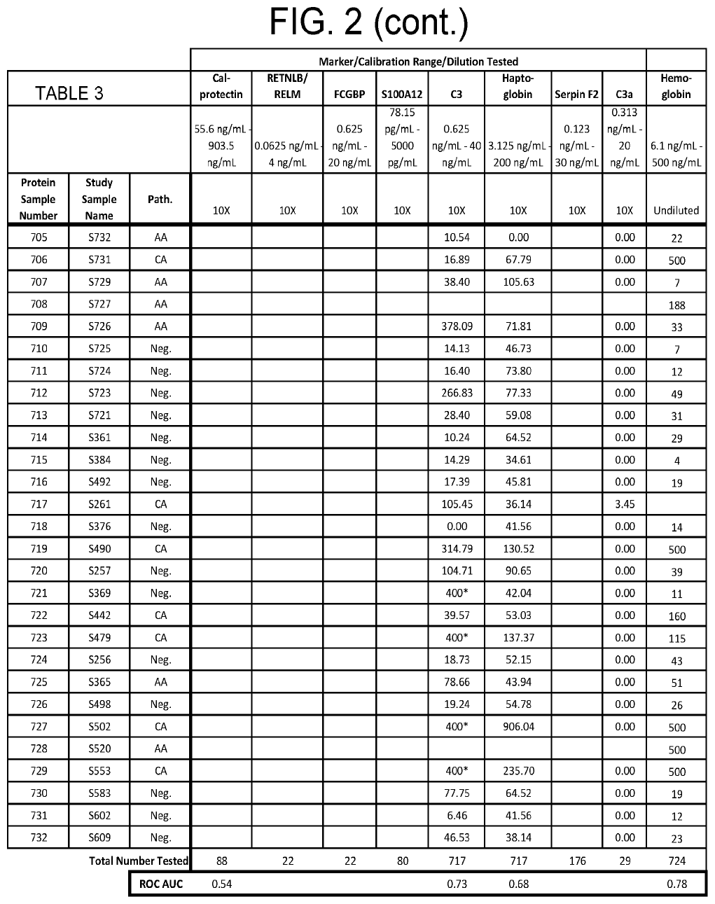

The samples chosen for the evaluation consisted of a population with known pathology. Not all samples were evaluated with each protein assay. A list of the protein markers that were evaluated in the whole stool supernatant samples and their results are found in Table 2 (FIG. 1). Protein markers that were evaluated in FIT device samples and their results are found in Table 3 (FIG. 2).

Of the 24 protein biomarkers evaluated, Haptoglobin (Hp) and Complement Component 3 (C3), when combined with the Hb result, showed improved sensitivity and specificity over the FIT for Hb alone. These two biomarkers demonstrated the most potential at improving the sensitivity and specificity for FIT sample testing when combined with Hemoglobin result and were tested on a large panel of samples with known pathologies. The sample panel consisted of 115 cancer samples (CA), 112 advanced adenoma samples (AA), and 488 Negative for Cancer. Whole stool samples were collected and a small portion of each was removed with a FIT collection device so that results from whole stool supernatant testing could be compared to the corresponding small sample processed using the FIT collection device.

To evaluate the performance of each biomarker to detect cancer, ROC curve analysis was performed, and the area under the curve (AUC) was calculated.

Table 4 shows AUC results from analysis of markers shown in FIG. 1 on whole stool supernatant samples. AUC result greater than 0.6 were selected for further analysis. Poor biomarker detection was observed in several cases and these markers were not included in AUC calculations.

TABLE 4
AUC Results from Whole Stool Supernatants

| Protein Biomarker | AUC Result |
|---|---|
| VWA2 | 0.44 |
| Gal 1 | 0.48 |
| LY6G6D | 0.52 |
| CA 19-9 | 0.53 |
| C3a des Arg | 0.54 |
| sIgA | 0.55 |
| PIGR | 0.56 |
| Calprotectin | 0.59 |
| CEA | 0.60 |
| FCGBP | 0.60 |
| Haptoglobin | 0.74 |
| C3 | 0.79 |
| Hemoglobin | 0.79 |
| C3a | NA |
| C5a | NA |
| CLDN1 | NA |
| Gal 3 | NA |
| GPR56 | NA |
| LRRC4 | NA |
| RETNLB/RELM | NA |
| Serpin F2 | NA |

Table 5 shows AUC results from analysis of markers shown in FIG. 2 on FIT device samples. AUC result greater than 0.6 were selected for further analysis. Poor biomarker detection was observed in several cases and these markers were not included in AUC calculations.

TABLE 5
AUC Results from FIT Device Samples

| Protein Biomarker | AUC Result |
|---|---|
| Calprotectin | 0.54 |
| Haptoglobin | 0.68 |
| C3 | 0.73 |
| Hemoglobin | 0.78 |
| C3a | NA |
| FCGBP | NA |
| RETNLB/RELM | NA |
| S100A12 | NA |
| Serpin F2 | NA |

Whole stool and corresponding FIT collection device small samples were tested with the C3 and Hp assays. The C3 and Hp results were modeled and the specificity and sensitivity for each individual assay, and for the assays in combination with the hemoglobin results from samples collected with the FIT collection device.

### Whole Stool Supernatant Samples

The results for the whole stool supernatant samples are shown in Table 6, below. These data show that in whole stool samples, the C3 marker alone demonstrated improved cancer sensitivity compared to the hemoglobin marker, and the greatest sensitivity was achieved when the C3, Hp, and Hb markers were combined.

TABLE 6
Biomarkers tested on whole stool supernatants

| | 93% Specificity | | | |
|---|---|---|---|---|
| | AA | CA | AA/CA | |
| | Sens | Sens | Sens | Notes |
| Hb | 23.3% | 69.0% | 47.6% | |
| C3 | 23.2% | 73.0% | 48.3% | Improved sensitivity |
| Hp | 21.7% | 59.5% | 41.9% | No improvement |
| Hb/C3 | 26.7% | 84.5% | 57.3% | Improved sensitivity |
| Hb/Hp | 29.2% | 84.5% | 58.6% | Improved sensitivity |
| C3/Hp | 25.0% | 73.3% | 50.7% | Improved sensitivity |
| Hb/C3/Hp | 30.8% | 85.3% | 59.9% | Improved sensitivity |

### FIT Device Samples

In samples collected with the FIT collection device (i.e., samples of approximately 20 mg), the greatest sensitivity was achieved when the C3, Hp, and Hb results were combined, as shown in Table 7. These data show that overall sensitivity for analysis of the FIT sample improved when the Hb result was combined with the C3 or Hp results, as compared to the results obtained with the FIT Hb analysis alone.

TABLE 7
Biomarkers tested on samples collected
with the FIT collection device.

| | 93% Specificity | | | |
|---|---|---|---|---|
| | AA | CA | AA/CA | |
| | Sens | Sens | Sens | Notes |
| Hb | 25.8% | 76.7% | 52.9% | |
| C3 | 15.0% | 64.7% | 41.0% | No improvement |
| Hp | 19.2% | 61.2% | 41.4% | No improvement |
| Hb/C3 | 28.3% | 80.2% | 56.0% | Improved sensitivity |
| Hb/Hp | 25.8% | 76.7% | 52.9% | Improved sensitivity |
| C3/Hp | 15.0% | 64.7% | 41.0% | No improvement |
| Hb/C3/Hp | 28.3% | 80.2% | 55.9% | Improved sensitivity |

### Whole Stool Supernatant Sample Data+FIT Device Sample Data

Data collected from whole stool supernatants and corresponding FIT collection device sample data (i.e., FIT samples taken from the whole stool sample and processed in parallel) were also combined. Table 8 shows the AUC results for analysis of the C3, Hp, and Hb markers when FIT and whole stool supernatant data for that marker are combined:

TABLE 8
AUC Results from Combined FIT and Stool Supernatant results.

| Protein Biomarker | AUC Result |
|---|---|
| Hb | 0.80 |
| C3 | 0.79 |
| Hp | 0.76 |
| Hb/C3 | 0.85 |
| Hb/Hp | 0.82 |
| C3/Hp | 0.80 |
| Hb/C3/Hp | 0.85 |

When both the FIT sample and whole stool supernatant sample were tested in combination, the results showed generally improved sensitivity as compare to the tests of each individual marker in whole stool or FIT device sampling alone, as shown in Table 9, below. These data show that overall sensitivity for Hb was improved when both the FIT sample and corresponding whole stool sample were tested, and the results were combined. Testing of Hp and/or C3 in addition to Hb provided a slight further improvement in sensitivity over Hb analysis alone.

TABLE 9
Combined Biomarker FIT and Stool Supernatant Results

| | 93% Specificity | | | |
|---|---|---|---|---|
| | AA | CA | AA/CA | |
| | Sens | Sens | Sens | Notes |
| Hb | 31.3% | 87.5% | 59.4% | |
| C3 | 22.3% | 75.9% | 49.1% | No improvement |
| Hp | 24.1% | 66.1% | 45.1% | No improvement |
| Hb/C3 | 31.3% | 88.4% | 59.8% | Improved sensitivity |
| Hb/Hp | 32.1% | 87.5% | 59.8% | Improved sensitivity |
| C3/Hp | 27.7% | 75.0% | 51.3% | No improvement |
| Hb/C3/Hp | 33.9% | 86.6% | 60.3% | Improved sensitivity |

Assays as described above may be further enhanced by the addition of an assay to detect one or more other molecules. Those of skill in the art will appreciate that detection of protein markers in fecal samples, including FIT device samples and whole stool supernatant sample may be combined with the detection of other markers, for example, methylation marker gene(s) or other nucleic acids in stool samples, and/or markers in samples of blood or blood products, including but not limited to RNA(s), methylation marker gene(s) and/or autoantibody(ies), individually or in any combination, to further enhance overall sensitivity.

All literature and similar materials cited in this application, including but not limited to, patents, patent applications, articles, books, treatises, and internet web pages are expressly incorporated by reference in their entirety for any purpose. Unless defined otherwise, all technical and scientific terms used herein have the same meaning as is commonly understood by one of ordinary skill in the art to which the various embodiments described herein belongs. When definitions of terms in incorporated references appear to differ from the definitions provided in the present teachings, the definition provided in the present teachings shall control.

Various modifications and variations of the described compositions, methods, and uses of the technology will be apparent to those skilled in the art without departing from the scope and spirit of the technology as described. Although the technology has been described in connection with specific exemplary embodiments, it should be understood that the invention as claimed should not be unduly limited to such specific embodiments. Indeed, various modifications of the described modes for carrying out the invention that are obvious to those skilled in pharmacology, biochemistry, medical science, or related fields are intended to be within the scope of the following claims.

What is claimed is:

1. A method of characterizing a human fecal sample, comprising:
i) providing a dispersed sample comprising a small human fecal sample dispersed in a volume of a stabilizing solution,
ii) assaying the dispersed sample for amounts of a plurality of different marker analytes.

2. The method of claim 1, wherein the volume of stabilizing solution is less than 3 mL, preferably less than 2 mL.

3. The method of claim 1 or claim 2, wherein the plurality of different marker analytes comprises a plurality of different protein marker analytes.

4. The method of claim 3, wherein the plurality of different protein marker analytes comprises at least three different protein marker analytes.

5. The method of any one of claims 1-4, wherein the plurality of different marker analytes comprises no more than three different marker analytes.

6. The method of any one of claims 1-5, wherein the plurality of different marker analytes are human marker analytes.

7. The method of any one of claims 1-6 wherein the plurality of different marker analytes comprises a plurality of human protein marker analytes selected from the group consisting of hemoglobin (Hb), calprotectin, haptoglobin (Hp), complement component 3 (C3), complement component 3a (C3a), Fc fragment of IgG binding protein (FCGBP), resistin like beta (RETNLB/RELM), S100 calcium binding protein A12 (S100A12), and serpin family F member 2 (SERPIN F2).

8. The method of claim 7, wherein the plurality of different human protein marker analytes comprises Hb, Hp, and C3.

9. The method of claim 8, wherein the plurality of different human protein marker analytes consists of Hb, Hp, and C3.

10. The method of any one of claims 1-9, wherein the assaying comprises an immunochemical assay.

11. The method of any one of claims 1-10, wherein the small fecal sample has a mass of less than 100 mg, preferably less than 50 mg, preferably less than 20 mg.

12. The method of any one of claims 1-11, wherein the stabilizing solution comprises a buffer.

13. The method of any one of claims 1-12, wherein the stabilizing solution comprises one or more of:
tris(hydroxymethyl)aminomethane (Tris) buffer;
bovine serum albumin;
polyethylene glycol sorbitan monolaurate (TWEEN-20);
sodium azide;
sodium chloride;
ethylenediaminetetraacetic acid (EDTA);
gentamicin.

14. The method of claim 13, wherein the stabilizing solution comprises 20 mM Tris, 10% bovine serum albumen, 0.10% TWEEN 20, 0.095% sodium azide, 140 mM sodium chloride, 10 mM EDTA, and 15 μg/ml gentamicin.

15. The method of any one of claims 1-14, wherein the stabilizing solution comprises one or more stabilization reagents selected from:
a protoporphyrin;
a polyvalent cation;
a sugar or polysaccharide;
an osmolyte and optionally, a polyvalent cation;
a horse radish peroxidase (HRP) stabilization component and, optionally, a polyvalent cation.

16. The method of any one of claims 1-15, wherein the stabilizing solution comprises protoporphyrin IX complexed with a multivalent cation, preferably a multivalent cation selected from Cr³⁺ and Co³⁺.

17. The method of any one of claims 1-16, wherein the stabilizing solution comprises a substituted or unsubstituted polygalacturonic acid and, optionally, a polyvalent cation.

18. The method of any one of claims 1-17, wherein the dispersed sample is provided in a FIT sample collection device.

19. The method of any one of claims 1-18, wherein the small human fecal sample is provided by a method comprising
a) collecting a whole stool sample from a human subject;
b) removing a portion of the whole stool sample to produce a removed portion and a remaining portion of the stool sample, wherein the removed portion is a small fecal sample;
c) combining the small fecal sample with the volume of stabilizing solution to produce the dispersed sample; and
d) stabilizing the remaining portion of the stool sample.

20. The method of claim 19, wherein the stabilizing of the remaining portion of the stool sample comprises one or both of:
i) adding a stabilizing buffer to the remaining portion of the stool sample, preferably homogenizing the remaining portion of the stool sample in a stabilizing buffer to form a stool homogenate;
ii) freezing the remaining portion of the stool sample.

21. The method of claim 20, wherein the stabilizing buffer comprises about 100 to about 300 mM of a chelating agent, preferably a chelating agent comprising EDTA, and comprises between about 400 and about 600 mM of Tris hydrochloride.

22. The method of claim 20 or 21, further comprising assaying the remaining portion of the stool sample for amounts of a one or more marker analytes.

23. The method of claim 22, wherein assaying the remaining portion of the stool sample comprises processing a stool homogenate to separate solids from a clarified fluid fraction and assaying the clarified fluid fraction.

24. The method of claim 23, wherein the processing the stool homogenate comprises one or more of filtration and centrifugation.

25. The method of any one of claims 22-24, wherein the one or more marker analytes assayed in the remaining portion of the stool sample comprises a marker analyte assayed in the dispersed sample.

26. The method of any one of claims 22-25, wherein the one or more marker analytes assayed in the remaining portion of the stool sample comprises the plurality of marker analytes assayed in the dispersed sample.

27. The method of any one of claims 22-26, wherein the one or more marker analytes assayed in the remaining portion of the stool sample consists of the plurality of marker analytes assayed in the dispersed sample.

28. The method of any one of claims 22-26, wherein the one or more marker analytes assayed in the remaining portion of the stool sample consists of marker analytes not assayed in the dispersed sample.

29. The method of any one of claims 14-26 and 28, wherein the plurality of different marker analytes assayed in the remaining portion of the stool sample comprises a plurality of human protein marker analytes selected from the group consisting of Hb, calprotectin, Hp, C3, C3a, FCGBP, RETNLB/RELM) S100A12, and SERPIN F2.

30. The method of claim 29, wherein the plurality of different human protein marker analytes assayed in the remaining portion of the stool sample comprises Hb, Hp, and C3.

31. The method of claim 25, wherein the plurality of different human protein marker analytes assayed in the remaining portion of the stool sample consists of Hb, Hp, and C3.

32. The method any one of claims 22-26 and 28-30, wherein the plurality of different marker analytes assayed in the remaining portion of the stool sample comprises one or more human DNA marker analytes.

33. The method of claim 32, wherein the one or more human DNA marker analytes are assayed for at least one of:
a mutation; and
a methylation status of a cytosine.

34. A set of articles for characterizing a human fecal sample, comprising:
i) a FIT sample collection device containing a dispersed sample comprising a small human fecal sample dispersed in a volume of a stabilizing solution,
ii) a set of reagents for assaying the dispersed sample for amounts of a plurality of different human protein marker analytes.

35. The set of articles of claim 34, wherein the volume of stabilizing solution in the FIT sample collection device is less than 3 mL, preferably less than 2 mL.

36. The set of articles of claim 34 or 35, wherein the plurality of different human protein marker analytes comprises at least three different protein marker analytes.

37. The set of articles of any one of claim 34-36, wherein the plurality of different human protein marker analytes comprises no more than three different marker analytes.

38. The set of articles of any one of claims 34-37, wherein the plurality of different human protein marker analytes is selected from the group consisting of Hb, calprotectin, Hp, C3, C3a, FCGBP, RETNLB/RELM) S100A12, and SERPIN F2.

39. The set of articles of claim 38, wherein the plurality of different human protein marker analytes comprises Hb, Hp, and C3.

40. The set of articles of claim 39, wherein the plurality of different human protein marker analytes consists of Hb, Hp, and C3.

41. The set of articles of any one of claims 34-40, wherein the set of reagents for assaying the dispersed sample comprises a set of reagents for immunochemical assays.

42. The set of articles of any one of claims 34-41, wherein the small human fecal sample has a mass of less than 100 mg, preferably less than 50 mg, preferably less than 20 mg.

43. The set of articles of any one of claims 34-42, wherein the stabilizing solution comprises a buffer.

44. The set of articles of any one of claims 34-43, wherein the stabilizing solution comprises one or more of:
Tris buffer;
bovine serum albumin;
TWEEN-20;
sodium azide;
sodium chloride;
EDTA;
gentamicin.

45. The set of articles of claim 44, wherein the stabilizing solution comprises 20 mM Tris, 10% bovine serum albumen, 0.10% TWEEN 20, 0.095% sodium azide, 140 mM sodium chloride, 10 mM EDTA, and 15 μg/ml gentamicin.

46. The set of articles of any one of claims 34-45, wherein the stabilizing solution comprises one or more stabilization reagents selected from:
a protoporphyrin;
a polyvalent cation;
a sugar or polysaccharide;
an osmolyte and optionally, a polyvalent cation;
a horse radish peroxidase (HRP) stabilization component and, optionally, a polyvalent cation.

47. The set of articles of any one of claims 34-46, wherein the stabilizing solution comprises protoporphyrin IX complexed with a multivalent cation, preferably a multivalent cation selected from Cr³⁺ and Co³⁺.

48. The set of articles of any one of claims 34-47, wherein the stabilizing solution comprises a substituted or unsubstituted polygalacturonic acid and, optionally, a polyvalent cation.

49. The set of articles of any one of claims 34-48, wherein the small human fecal sample comprises a portion removed from a whole stool sample, wherein the set of articles further comprises a stabilized remaining portion of the stool sample.

50. The set of articles of claim 49, wherein the stabilized remaining portion of the stool sample comprises a stabilizing buffer.

51. The set of articles of claim 50, wherein the stabilized remaining portion of the stool sample the stabilizing buffer comprises about 100 to about 300 mM of a chelating agent, preferably a chelating agent comprising EDTA, and comprises between about 400 and about 600 mM of Tris hydrochloride.

52. The set of articles of claim 50 or 51, wherein in the stabilized remaining portion of the stool sample, the remaining portion of the stool sample is homogenized in the stabilizing buffer.

53. The set of articles of claim 49, further comprising a set of reagents for assaying the stabilized remaining portion of the stool sample for amounts of one or more marker analytes.

54. The set of articles of claim 53, wherein the one or more marker analytes assayable by the set of reagents for assaying the stabilized remaining portion of the stool sample comprises at least one human protein marker analyte assayable by the set of reagents for assaying the dispersed sample.

55. The set of articles of claim 53, wherein the one or more marker analytes assayable by the set of reagents for assaying the stabilized remaining portion of the stool sample comprises one or more human DNA marker analytes.

56. The set of articles of claim 55, wherein the set of reagents for assaying the stabilized remaining portion of the stool sample for one or more human DNA marker analytes comprises reagents for assaying at least one of:
a mutation; and
a methylation status of a cytosine.
